(19) United States
(12) Patent Application Publication
(10) Pub. No.: US 2023/0329944 A1
(43) Pub. Date: Oct. 19, 2023

(54) SHIELDED CHAMBER FOR DIAGNOSTIC EVALUATION OF MEDICAL CONDITIONS

(71) Applicant: Genetesis, Inc., Mason, OH (US)

(72) Inventors: Vineet Naveen ERASALA, Mason, OH (US); Emmanuel T SETEGN, Mason, OH (US); Peeyush SHRIVASTAVA, Mason, OH (US); Rhea MALHOTRA, Mason, OH (US); Yosef KIRSCHNER, Mason, OH (US)

(21) Appl. No.: 18/116,965

(22) Filed: Mar. 3, 2023

Related U.S. Application Data
(60) Provisional application No. 63/316,541, filed on Mar. 4, 2022.

(51) Int. Cl. A61G 10/00; A61B 5/242

(52) U.S. Cl. CPC A61G 10/005; A61B 5/242

(57) ABSTRACT

Disclosed herein is a shielded chamber system for diagnostic evaluation of a condition of an individual. The shielded chamber system comprises an enclosure comprising a plurality of walls, a wall comprising a plurality of layers of magnetic shielding material. The shielded chamber system also comprises one or more application-specific modular components configured to be inserted within the enclosure, wherein an application-specific modular component comprises an array of biomagnetic field sensors configured to sense an electromagnetic field associated with the individual and generate electromagnetic field data therefrom. Finally, the shielded chamber system comprises one or more holes or passthroughs inserted into at least one wall of the plurality of walls, wherein a hole or passthrough is configured for passing electrical or data cabling into and out of the enclosure.

### CROSS-REFERENCE

This application claims priority to U.S. Provisional Application No. 63/316,541, filed Mar. 4, 2022, which is entirely herein incorporated by reference.

### BACKGROUND

Dynamic magnetic fields are associated with certain mammalian tissue, for example, tissue with action-potential driven physiology. Changes in the structure or function of certain tissue can be reflected in a change of the magnetic field(s) associated with and/or generated by the tissue. Many medical centers and individual healthcare providers utilize computer based systems for biomagnetic detection and analysis of patient data.

### SUMMARY

The present disclosure provides systems, devices, and methods for sensing a magnetic field such as an electromagnetic field (“EMF”), magnetoencephalogram (“MEG”) or a magnetocardiogram (“MCG”) associated with a tissue of an individual, a portion of a body of an individual, and/or an entire body of an individual. Non-limiting examples of tissue for which a magnetic field is associated and sensed using the systems, devices, and methods described herein include blood, bone, lymph, cerebrospinal fluid (CSF), and organs including the heart, lungs, liver, kidneys, and skin. In some embodiments, the devices and systems described herein sense a magnetic field signal associated with a portion of a body of an individual, such as, for example a torso of an individual, or a magnetic field associated with the entire body of the individual.

In an aspect, a shielded chamber system for diagnostic evaluation of a condition of an individual is disclosed. The shielded chamber system comprises an enclosure comprising a plurality of walls, a wall comprising a plurality of layers of magnetic shielding material. The shielded chamber system also comprises one or more application-specific modular components configured to be inserted within the enclosure, wherein an application-specific modular component comprises an array of biomagnetic field sensors configured to sense an electromagnetic field associated with the individual and generate electromagnetic field data therefrom. The shielded chamber system also comprises one or more holes or passthroughs inserted into at least one wall of the plurality of walls, wherein a hole or passthrough is configured for passing electrical or data cabling into and out of the enclosures.

In some embodiments, the wall comprises two or more layers.

In some embodiments, each of the two or more layers has a thickness of between 0.1 and 10 millimeters.

In some embodiments, the wall comprises a permalloy or a mumetal.

In some embodiments, the wall comprising a permalloy or a mumetal is built around a nonmagnetic frame.

In some embodiments, one of the one or more application-specific modular components is directed to cardiac applications.

In some embodiments, one of the one or more application-specific modular components is a magnetocardiography (“MCG”) module.

In some embodiments, one of the one or more application-specific modular components is directed to neurological applications.

In some embodiments, one of the one or more application-specific modular components is a magnetoencephalography (“MEG”) module.

In some embodiments, one of the one or more application-specific modular components is a module for magnetorelaxometry, employing magnetization coils for site-specific magnetorelaxometry measurements

In some embodiments, one of the one or more application-specific modular components is a module for ultra-low field magnetic resonance imaging (“MRI”) employing magnetization coils to produce an image of the individual.

In some embodiments, the shielded chamber system comprises a mounting system comprising the one or more application-specific modular components.

In some embodiments, the array of biomagnetic field sensors are actuated to create a multi-frame stitched data image.

In some embodiments, the array of biomagnetic field sensors comprises at least three biomagnetic field sensors.

In some embodiments, the array of biomagnetic field sensors is arranged to match a generalized contour of a portion of a body of the individual.

In some embodiments, the array of biomagnetic field sensors comprises optically pumped magnetometer sensors, magnetic induction sensors, magneto-resistive sensors, SQUID sensors, nitrogen vacancy diamonds, fluxgate magnetometers, or a combination thereof.

In some embodiments, the fluxgate magnetometers comprise Yttrium Iron Garnet film.

In some embodiments, one of the one or more application-specific modular components is a module for fetal magnetocardiography.

In some embodiments, one of the one or more application-specific modular components is a module for fetal magnetoencephalography.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a schematic of a magnetically shielded room or chamber.

FIG. 2 shows a photograph of a magnetically shielded chamber or room with the shielded door opened.

FIG. 3 shows a computer system that is programmed or otherwise configured to implement methods provided herein.

FIG. 4 shows an example of a sensor array.

FIG. 5 shows an example of a 3D rendering of a sensor head cage mounted on a bed of a shield.

FIG. 6 shows an exemplary layout of one inner coil positioned in an embodiment of a shield.

FIG. 7 shows an exemplary layout of outer coils positioned in an embodiment of a shield.

FIG. 8 shows a typical equilibration function.

FIG. 9 depicts an example environment that can be employed to execute implementations of the present disclosure.

FIG. 10 depicts an example platform architecture that can be employed according to implementations of the present disclosure.

FIG. 11 depicts a schematic representation of an exemplary medical device that can be employed according to implementations of the present disclosure.

FIGS. 12A-12B depict schematic examples of neural network architecture in terms of flow of data within the neural network.

FIG. 13 shows a hook configured to span a portion of or an entire volume of a shield.

FIG. 14 depicts a schematic representing an exemplary machine learning software module.

### DETAILED DESCRIPTION

While various embodiments are shown and described herein, it will be obvious to those skilled in the art that such embodiments are provided by way of example only. It should be understood that various alternatives to the embodiments herein are employed.

As used herein, the singular forms “a”, “an”, and “the” include plural references unless the context clearly dictates otherwise. Any reference to “or” herein is intended to encompass “and/or” unless otherwise stated.

Described herein is a platform that includes a set of hardware and software tools employed to capture, analyze, and report results from collected patient magnetic fields. In some embodiments, a platform as described herein includes an EMF sensing system which further includes one or more hardware (device(s)) and software. In some embodiments, a platform as described herein comprises at least one health care provider portal and a server configured to provide at least one healthcare related service.

In some embodiments, the described platform is employed to provide results quickly, (e.g., within one hour) after an EMF scan is taken. Results may include suggesting further testing or a definitive ruling out of a patient. In some embodiments, the described platform is employed to reduce hospital burden with low to intermediate risk patients as well as streamlining certain administrative or healthcare finance tasks such as, for example, billing or insurance form submission.

In some embodiments, the described platform is deployed as a service (PaaS) and cognitive engine employed to unify a set of disjointed services in, for example, a hospital to streamline medical device usage process. In some embodiments, the described platform performs functions, such as ordering, scanning, image and signal processing, reader image analysis, and reporting. These functions can be broadly extended to many medical devices deployed in a hospital setting to collect a wide array of unique signals, e.g., ECG, magnetocardiography, magnetoencephalography, magnetic resonance imaging (MRI), computerized tomography (CT), and so forth. In some embodiments, devices are preconfigured to interact with RESTful API services provided through the employed PaaS. In some embodiments, devices are connected to an existing Electronic Health Record (EHR) system to associate scans taken with a respective patient. For example, in some embodiments, when a scan is completed, a device uploads the data to the employed PaaS for processing and storage. In some embodiments, the data is analyzed by a healthcare provider who has access to the set of signals, images and tools used to analyze different types of signals or images. In some embodiments, once decided on scan quality, diagnosis, and noting any other additional comments, the healthcare provider may submit a report that is then accessible by, for example, an ordering healthcare provider, with patient demographics, scan information, signal and image metrics and parameters, and a machine-learning based score.

In various embodiments, the platforms, systems, media, and methods described herein include a cloud computing environment. In some embodiments, a cloud computing environment comprises one or more computing processors.

While various embodiments are shown and described herein, it will be obvious to those skilled in the art that such embodiments are provided by way of example only. It should be understood that various alternatives to the embodiments herein in some embodiments are employed.

As used in this specification and the appended claims, the singular forms “a,” “an,” and “the” include plural references unless the context clearly dictates otherwise. Any reference to “or” herein is intended to encompass “and/or” unless otherwise stated.

As used herein, the phrases “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

In general, the term “software” as used herein comprises computer readable and executable instructions that may be executed by a computer processor. In some embodiments a “software module” comprises computer readable and executable instructions and may, in some embodiments described herein make up a portion of software or may in some embodiments be a stand-alone item. In various embodiments, software and/or a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

A “managed physician” includes a user on the described platform that is to read and interpret results received from, for example, an EMF sensing device or system.

A “magnetocardiogram” or “MCG” is a visual representation of the magnetic fields produced by the electrical activity of the heart. An MCG as used herein includes an MCG generated from any technique that determines one or more magnetic fields associated with a heart of an individual including techniques as described herein using one or more EMF sensors as well as traditional magnetic resonance imaging techniques. A “CardioFlux” is a brand name of a system such as the systems described herein that is configured to sense an EMF associated with a patient and in some embodiments uses the sensed EMF to generate an MCG or other visual representation of an EMF. A CardioFlux system, in some embodiments, includes or is operatively coupled to software configured to analyze a sensed EMF and in some embodiments is configured to determine a diagnosis of a patient based on a sensed EMF from the patient.

“Magnetoencephalography” or “MEG” is a functional neuroimaging technique for mapping brain activity by recording magnetic fields produced by electrical currents occurring naturally in the brain. An MEG as used herein includes an MEG generated from any technique that determines one or more magnetic fields associated with brain activity of an individual including techniques as described herein.

“Amazon Web Services” or “AWS” is an on demand cloud computing platform.

A “global reader portal” or “GRP” is a user portal in a platform as described herein and in some embodiments provides a managed physician with the ability to view medical data including, for example, one or more medical images and provide one or more interpretations of the one or more medical images.

A “site reader portal” or “SRP” is a user portal in a platform as described herein and in some embodiments provides authorized site users with the ability to view medical data including, for example, raw medical data, interpretation results, and/or patient demographic information.

An “application programming interface” or “API” includes a set of subroutine definitions, communication protocols and tools for building software. In some embodiments, an API provides an authorized user the ability to integrate software into a platform as described herein in order to, for example, customize one or more features of the platform.

“Microservices” are a software architecture style in which complex applications are composed of small independent processes communicating with each other, using language agnostic APIs.

An “API Gateway” is an exposed set of one or more API endpoints that coordinate a set of calls to different microservices.

“Representational State Transfer” or “REST” is an architectural style that defines a set of constraints to be used for creating web services and provides interoperability between computer systems and the Internet.

“JSON Web Token” or “JWT Token” is a JSON-based open standard (RFC 7519) for creating access tokens that assert some number of claims and may include user information including encrypted user information.

“Electromagnetic field” or “EMF” data includes EMF measurements and simulations of EMF measurements.

### Devices and Systems for Sensing a Magnetic Field

Described herein are devices and systems configured to sense a magnetic field associated with one or more tissues, one or more body portions, one or more organs, or an entire body of an individual. Non-limiting examples of organs and organ systems having a magnetic field that is sensed by the devices and systems described herein include the brain, heart, lungs, kidneys, liver, spleen, pancreas, esophagus, stomach, small bowel, and colon, the endocrine system, respiratory system, cardiovascular system, genitourinary system, nervous system, vascular system, lymphatic system, and digestive system. Non-limiting examples of tissue having a magnetic field that is sensed by the devices and systems described herein includes inflammatory tissue (including areas of inflamed tissue), blood vessels and blood flowing within blood vessels, lymphatic vessels and lymph flowing within lymphatic vessels, bone, and cartilage. Magnetic field data that is sensed is further processed in order to make determinations or assist a user (e.g. a healthcare provider) in making a determination about the one or more tissues, the one or more body portions, the one or more organs, or the entire body of the individual that is associated with that sensed magnetic field. For example, in some embodiments, a device as described herein is used to determine a prognosis of an individual, such as, for example, predicting a likelihood of an individual developing a disease or condition based on one or more magnetic fields that are sensed using the device. For example, in some embodiments, a device as described herein is used to determine a diagnosis, such as, for example, confirming a diagnosis or providing a diagnosis to an individual for a disease or condition based on one or more magnetic fields that are sensed using the device. For example, in some embodiments, a device as described herein is used to provide monitoring, such as monitoring a progression of a disease or condition in an individual, monitoring an effectiveness of a therapy provided to an individual, or a combination thereof based one or more magnetic fields that are sensed using the device. It should be understood that the devices and systems described herein are suitable for measuring a magnetic field associated with any type of tissue.

In some embodiments of the devices and systems described herein, sensed magnetic field data associated with a heart is used to generate a magnetocardiogram. In these embodiments of the devices and systems described herein, the devices and systems are utilized as a magnetocardiograph which is, for example, a passive, noninvasive bioelectric measurement tool intended to detect, record, and display magnetic fields that are naturally generated by electrical activity of a heart.

In some embodiments, a device or system as described herein is configured to measure one or more biomarkers in addition to a magnetic field. Non-limiting examples of biomarkers sensed in addition to a magnetic field using embodiments of the devices and systems described herein include a body temperature, a heart rate, blood pressure, an echocardiogram (ECG), a magnetic field, or any combination thereof.

In some embodiments, an individual, whose magnetic field is sensed, is in good health. In some embodiments, an individual, whose magnetic field is sensed, is an individual suspected of having a condition or disease. In some embodiments, an individual, whose magnetic field is sensed, is an individual having received a previous diagnosis of having a condition or disease.

In some embodiments, a condition or disease being identified in an individual is a cardiac condition or disease. In some embodiments, a cardiac condition or disease being identified in an individual comprises rheumatic heart disease, hypertensive heart disease, ischemic heart disease, cerebrovascular disease, inflammatory heart disease, valvular heart disease, an aneurysm, a stroke, atherosclerosis, arrhythmia, hypertension, angina, coronary artery disease, coronary heart disease, a heart attack, cardiomyopathy, pericardial disease, congenital heart disease, heart failure, or any combination thereof.

In some embodiments, a condition or disease being identified in an individual is a neurological disease. In some embodiments, the systems, methods, devices, and software described herein are used to evaluate an individual for neurological disease including abnormalities resulting from traumatic injury and stroke. Non-limiting examples of neurological disorders evaluated by the systems, methods, devices, and software described herein include epilepsy, concussion, stroke, traumatic brain injury, traumatic spine injury, encephalitis, meningitis, tumor, Alzheimer's disease, Parkinson's disease, ataxia, and psychiatric disorders including schizophrenia, depression, and bipolar disease.

A device as described herein, in some embodiments, comprises one or more sensors. In some embodiments, two or more sensors are arranged in a sensor array. In some embodiments, a device as described herein includes an electromagnetic shield, and some embodiments of the devices described herein do not include a shield.

Systems as described herein, in some embodiments, comprise any device as described herein and one or more local and/or remote processors.

### Sensors and Sensor Arrays for Sensing a Magnetic Field

In some embodiments of the devices and systems described herein, a device comprises a sensor, such as an optically pumped magnetometer (OPM) as a measurement tool, which, in some embodiments, utilizes nonradioactive self-contained alkali metal cells coupled with a closed pumping laser and photodetector setup to measure minute magnetic fields. In some embodiments of the devices and systems described herein, the devices and systems utilize OPMs in an n×n array (or grid) or alternative geometric configuration to collect magnetic field data at n discrete locations over, for example, a portion of a body of an individual such as a chest area, which, in some embodiments, is digitized using pickup electronics.

OPMs are typically configured to utilize nonradioactive self-contained alkali metal cells coupled with a closed pumping laser and photodetector setup to measure minute magnetic fields. Compared to superconducting quantum interference devices (SQUIDs), which are typically also used to detect these biomagnetic fields, OPM sensors are significantly smaller and typically do not require the use of cryogenic cooling.

The Earth's magnetic field is naturally present everywhere on Earth, and the amplitude is about 50 microtesla. OPM performance is enhanced in at least two exemplary ways in the presence of the Earth's ambient magnetic field. In a first OPM enhancing technique, a reference value representing Earth's magnetic field is used as part of a vector subtraction to isolate a signal of interest in an OPM. Another technique involves the use of a gradiometer for active noise cancellation for the OPM.

A sensor array configuration, as utilized in some embodiments of the devices and systems described herein, comprises a custom array configuration. In some embodiments, a sensor array configuration is customized to an individual's anatomy. In some embodiments, a sensor array configuration is customized to a location on the individual which is measured, such as a chest location or a head location. In some embodiments, a sensor array configuration is customized to a measurement type that a device is programmed to acquire. In some embodiments, a sensor array configuration is customized to be operatively coupled with a shield and/or an arm. In some embodiments, a sensor array configuration is interchangeable with a different array configuration—a user may perform with interchange. An array configuration, in some embodiments, comprises an arc (such as a generally curved shape) having a depth and comprising a radius from about 20 cm to about 50 cm or from about 10 cm to about 60 cm. An array configuration, such as an arc configuration, in some embodiments, comprises one or more variable inter-magnetometer distances and variable sensor densities. An array configuration, in some embodiments, comprises a concave structure (such as a concave structure configured to wrap or form around a body region, such as a head or chest). One or more magnetometers is positioned on at least a portion of a surface of the concave structure. A concave array configuration, in some embodiments, comprises one or more variable inter-magnetometer distances and variable sensor density.

In some embodiments, a sensor array comprises n×n sensors. In some embodiments, a sensor array is a 2D rectangular array, such as a 2×2 array or a 4×4 array. In some embodiments, a sensor array is a 2D non-rectangular array, such as a 2×1 array or a 4×1 array. In some embodiments, a sensor array is a circular array or a semicircular array, such as a 3D array of sensors positioned in an arc or concave structure. In some embodiments, a sensor array is a 2D array or a 3D array. In some embodiments, a sensor of a sensor array comprises x, y, and z coordinates. An array, in some embodiments, comprises a single sensor, such as n×n=1×1. An array, in some embodiments, comprises two sensors, such as n×n=2×1. An array, in some embodiments, comprises three sensors. An array, in some embodiments, comprises four sensors. An array, in some embodiments, comprises nine sensors. An array, in some embodiments, comprises sixteen sensors. An array, in some embodiments, comprises 25 sensors. An array, in some embodiments, comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 sensors or more. In some embodiments, a sensor array comprises 8 sensors. In some embodiments, a sensor array comprises 16 sensors. In some embodiments, a sensor array comprises a single sensor housed in a single housing. In some embodiments, a sensor array comprises a plurality of sensors housed in a single housing, such as a housing having multiple sensor configurations or changeable sensor configurations. In some embodiments, a sensor array comprises a plurality of sensors housed in a plurality of housings. In some embodiments, a sensor array comprises a plurality of sensors, each sensor housed in a separate housing. In some embodiments, a first sensor and second sensor of a sensor array is different. In some embodiments, a first sensor and a second sensor of a sensor array is the same. In some embodiments, each sensor of a sensor array is unique. In some embodiments, each sensor of a sensor array is identical. In some embodiments, a subset of sensors within a sensor array is unique. In some embodiments, a subset of sensors within a sensor array is identical. Spatial positioning of a sensor in a sensor array is adjustable, such as by a user or automated by a controller. In some embodiments, spatial positioning of a sensor in a sensor array is fixed. In some embodiments, a number of sensors in a sensor array is selected based on an application. In some embodiments, a number of sensors in a sensor array is selected based on a type of measurement or a location of a measurement. An array, in some embodiments, comprises a single channel array or a multi-channel array. In some embodiments, increasing a number of sensors of a sensor array increases a resolution of a measurement taken by the array. In some embodiments, a sensor array of sensors is densely packed, such as substantially adjacent or proximal one another. An array of sensors is sparsely spaced, such as having a spacing between one another. In some embodiments, a subset of sensors of a sensor array is densely packed. In some embodiments, a subset of sensors of a sensor array is sparsely spaced or densely spaced. In some embodiments, centerpoints of any two sensors of a densely packed subset of sensors is spaced less than about: 5, 4.5, 4, 3.5, 3, 2.5, 2, 1.5, 1, 0.5, 0.1 centimeters (cm) apart. In some embodiments, centerpoints of densely packed sensors is spaced centerpoint to centerpoint from about 0.1 cm to about 2.0 cm or from about 0.1 cm to about 1.5 cm or from about 1.0 cm to about 2.0 cm. In some embodiments, centerpoints of any two sensors of a sparsely packed subset of sensors is spaced more than about: 1.5, 2, 2.5, 3, 3.5, 4, 4.5, 5, 8, 10 cm apart. In some embodiments, centerpoints of sparsely packed sensors is spaced centerpoint to centerpoint from about 1.5 cm to about 3 cm or from about 2 cm to about 5 cm or from about 2.5 cm to about 8 cm. In some embodiments, a center point is a central location of a sensor, such as a central axis. In some embodiments, a centerpoint of a circular sensor is a central point at which all other edge points are of equal distance.

In some embodiments, a densely packed array indicates intermagnetometer placement of less than 1.5 cm, while magnetometer placement of greater than about 1.5 cm constitutes a sparsely packed array.

In some embodiments, a housing is configured to house a sensor or a sensor array of sensors. In some embodiments, the housing is configured to accommodate a single configuration of sensor spacing within the housing. In some embodiments, the housing is configured to accommodate multiple configurations of sensor spacing within the housing. In some embodiments, the housing accommodates (i) adjusting sensor spacing, such as a dense spacing or a sparse spacing, or (ii) varying a number of sensors within the array. In some embodiments, a housing is a universal housing for a plurality of arrays and array configurations.

In some embodiments, a sensor is configured to sense a presence of or measure a parameter of a magnetic field. A sensor, in some embodiments, comprises a sensitivity to a magnetic field of about 10 femtotesla per root Hertz (fT/√Hz). A sensor, in some embodiments, comprises a sensitivity of from about 1 fT/√Hz to about 20 fT/√Hz. A sensor, in some embodiments, comprises a sensitivity of from about 5 fT/√Hz to about 15 fT/√Hz. A sensor, in some embodiments, comprises a sensitivity of from about 0.1 fT/√Hz to about 30 fT/√Hz. A sensor, in some embodiments, comprises a sensitivity of from about 0.5 fT/√Hz to about 12 fT/√Hz. A sensor, in some embodiments, comprises a sensitivity of from about 1 fT/√Hz to about 15 fT/√Hz. A sensor, in some embodiments, comprises a sensitivity of about: 0.001, 0.005, 0.01, 0.05, 0.1, 0.5, 1, 2, 3, 4, 5, 6, 7, 8, 10, 11, 12, 13, 14, 15, 16, 17, 18, 19, 20 fT/√Hz.

In some embodiments, a sensor does not require a cooling element, such as cryogenic cooling, to collect a measurement. In some embodiments, a sensor collects a measurement over a temperature range of from about 30 degrees Fahrenheit (F) to about 110 degrees F. In some embodiments, a sensor collects a measurement over a temperature range of from about 50 degrees F. to about 110 degrees F. In some embodiments, a sensor collects a measurement over a time period of from about 1 second to about 5 hours without a need for a cooling element. In some embodiments, a sensor collects a measurement over a time period of from about 1 second to about 1 hour without a need for a cooling element. In some embodiments, a sensor collect a measurement over a time period of from about 1 second to about 30 minutes without a need for a cooling element.

A noise source, in some embodiments, comprises a magnetic field strength. In some embodiments, a strength of a magnetic field of a noise source is measured in units of Tesla (T). Noise, such as ambient noise, in some embodiments, comprises a magnetic field strength of less than about 100 nanotesla (nT). Noise, in some embodiments, comprises a magnetic field strength of less than about 1000 nT. Noise, in some embodiments, comprises a magnetic field strength of less than about 500 nT. Noise, in some embodiments, comprises a magnetic field strength of less that about 200 nT. Noise, in some embodiments, comprises a magnetic field strength of less than about 120 nT. Noise, in some embodiments, comprises a magnetic field strength of less than about 80 nT. A noise source, such as a magnetic field of the Earth, in some embodiments, comprises a magnetic field strength of about 50 microtesla (mT). Noise, in some embodiments, comprises a magnetic field strength of from about 40 mT to about 60 mT. Noise, in some embodiments, comprises a magnetic field strength of from about 10 mT to about 100 mT. Noise, in some embodiments, comprises an amplitude component, a frequency component, or a combination thereof, and, in some embodiments, comprises both sources that is direct current (DC), alternating current (AC), or a combination of the two.

In some embodiments, the shielded room or chamber utilizes a raster framework. In some embodiments, the raster framework comprises sensors within an array. In some embodiments, the sensors within an array are actuated or moved to create multi-frame stitched data rather than a single shot capture of data. In some embodiments, the sensors within an array are actuated or moved to multiple positions wherein data is captured in several discrete steps and combined to create a multi-frame image and data series rather than a single-frame image. In some embodiments, the utilization of a raster framework expands coverage area of a sensor. In some embodiments, the utilization of a raster framework minimizes the amount of sensors required for a specific application. In some embodiments, a time gating device common to all data capture segments is utilized to synchronize timestamps for data analysis.

### Electromagnetic Shield

Some embodiments of the devices and systems as described herein are configured to provide an electromagnetic shield to reduce or eliminate the ambient magnetic field of the Earth. A shield as described herein, in some embodiments, comprises a metal alloy (e.g. permalloy or mumetal), which when annealed in a hydrogen furnace provides exceptionally high magnetic permeability, thereby isolating regions protected by the shield (e.g. within a shielded room or chamber) from the Earth's magnetic field.

A shield as described herein minimizes interior magnetic fields, and, in some embodiments, is constructed as a room or chamber. In some embodiments, the shielded room or chamber has walls. For example, the shielded room or chamber may have four walls. In some embodiments, the shielded room or chamber has a ceiling or roof. In some embodiments, the shielded room or chamber has a floor. In some embodiments, the shielded room or chamber has one or more doors. In some embodiments, when the door is shut, the shielded room or chamber is sealed off from the outside. In some embodiments, when the door is open, a patient can enter the shielded room or chamber.

In some embodiments, the shielded room or chamber comprises holes or passthroughs. In some embodiments, the holes and passthroughs allow electrical and data cabling to enter and exit the shielded room or chamber. In some embodiments, the shielded room or chamber may comprise a transparent window. In some embodiments, this window may allow a subject to be viewed and monitored from outside of the shielded room or chamber.

In some embodiments, the shielded room or chamber is built around a nonmagnetic frame. In some embodiments, this nonmagnetic frame comprises aluminum. In some embodiments, this nonmagnetic frame comprises aluminum, brass, copper, gold, lead, or silver.

In some embodiments, utilization of a shield with sensor, such as a sensor array provides a reduction of noise such that the sensor collects a measurement that is substantially free of a noise or collects a measurement in which a noise is significantly reduced. A noise, in some embodiments, comprises a noise from a noise source. In some embodiments, a noise source includes a high frequency noise, such as greater than about 20 Hz, a middle frequency noise, such as from about 1 Hz to about 20 Hz, a low frequency noise such as from about 0.1 Hz to about 1 Hz, or any combination thereof. In some embodiments, a noise source includes any structure comprising metal. In some embodiments, a structure comprising metal includes a metal implant such as a pacemaker, a defibrillator, an orthopedic implant, a dental implant, or others. In some embodiments, a structure comprising metal includes a metal tool, a metal door, a metal chair, or others. In some embodiments, a noise source includes operation of a device such as a fan, an air conditioner, a clinical apparatus, or vibrations of a building. In some embodiments, a noise source includes operation of a power supply or an electronic device such as a computer comprising a monitor or graphical user interface.

A shield or portion thereof, in some embodiments, comprises a single layer of material. A shield or portion thereof, in some embodiments, comprises a plurality of layers of a material. A shield or portion thereof, in some embodiments, comprises a plurality of layers, wherein at least two of a plurality of layers comprise different materials. A shield or portion thereof, in some embodiments, comprises 2 layers. A shield or portion thereof, in some embodiments, comprises 3 layers. A shield or portion thereof, in some embodiments, comprises 4 layers. A shield or portion thereof, in some embodiments, comprises 5 layers. A shield or portion thereof, in some embodiments, comprises 6 layers.

A layer of a shield or portion thereof, in some embodiments, comprises a thickness from about 0.1 to about 10 millimeters. In some embodiments, a layer of a shield has a thickness from about 0.5 to about 5 millimeters. In some embodiments, a layer of a shield has a thickness from about 0.1 to about 2 millimeters. In some embodiments, a layer of a shield has a thickness from about 0.8 to about 5 millimeters. A thickness is substantially the same along a length or a circumference of a shield. In some embodiments, a thickness of a layer of a shield varies along a length or circumference of a shield.

In some embodiments, a shield comprises a plurality of layers. In some embodiments, a space is present between at least two layers of the plurality of layers. In some embodiments, a space is present between each layer of the plurality of layers. In some embodiments, a space is present between a subset of layers of the plurality of layers. In some embodiments, a first layer of a shield is configured to be adjacent a second layer of a shield. In some embodiments, a first layer of a shield is configured to be attached or bonded to a second layer of a shield. In some embodiments, a first layer of a shield is configured to be positioned from about 0.1 inches to about 5 inches from a second layer. In some embodiments, a first layer of a shield is configured to be positioned from about 1 inch to about 3 inches from a second layer. In some embodiments, a first layer of a shield is configured to be positioned from about 1 inch to about 20 inches from a second layer. In some embodiments, a first layer of a shield is configured to be positioned from about 1 inch to about 10 inches from a second layer.

In some embodiments, the shield is a room or chamber. In some embodiments, the shielded room or chamber is configured to accommodate at least a portion of an individual. In some embodiments, the shielded room or chamber is configured to accommodate an individual. In some embodiments, the shielded room or chamber is configured to accommodate an individual and one or more mobility devices. In some embodiments, a mobility device is a manual wheelchair, power wheelchair, power scooter, hospital bed, crib, bassinet, stretcher, walker, cane, braces, or crutches. In some embodiments, an individual is a human subject. In some embodiments, a human subject is an adult subject, a pediatric subject, or a neonatal subject.

In some embodiments, the height of the shielded room or chamber is about 3 feet to about 15 feet. In some embodiments, the height of the shielded room or chamber is about 3 feet to about 5 feet, about 3 feet to about 6 feet, about 3 feet to about 7 feet, about 3 feet to about 8 feet, about 3 feet to about 9 feet, about 3 feet to about 10 feet, about 3 feet to about 15 feet, about 5 feet to about 6 feet, about 5 feet to about 7 feet, about 5 feet to about 8 feet, about 5 feet to about 9 feet, about 5 feet to about 10 feet, about 5 feet to about 15 feet, about 6 feet to about 7 feet, about 6 feet to about 8 feet, about 6 feet to about 9 feet, about 6 feet to about 10 feet, about 6 feet to about 15 feet, about 7 feet to about 8 feet, about 7 feet to about 9 feet, about 7 feet to about 10 feet, about 7 feet to about 15 feet, about 8 feet to about 9 feet, about 8 feet to about 10 feet, about 8 feet to about 15 feet, about 9 feet to about 10 feet, about 9 feet to about 15 feet, or about 10 feet to about 15 feet. In some embodiments, the height of the shielded room or chamber is about 3 feet, about 5 feet, about 6 feet, about 7 feet, about 8 feet, about 9 feet, about 10 feet, or about 15 feet. In some embodiments, the height of the shielded room or chamber is at least about 3 feet, about 5 feet, about 6 feet, about 7 feet, about 8 feet, about 9 feet, or about 10 feet. In some embodiments, the height of the shielded room or chamber is at most about 5 feet, about 6 feet, about 7 feet, about 8 feet, about 9 feet, about 10 feet, or about 15 feet.

In some embodiments, the width of the shielded room or chamber is about 2 feet to about 15 feet. In some embodiments, the width of the shielded room or chamber is about 2 feet to about 4 feet, about 2 feet to about 5 feet, about 2 feet to about 6 feet, about 2 feet to about 8 feet, about 2 feet to about 10 feet, about 2 feet to about 12 feet, about 2 feet to about 15 feet, about 4 feet to about 5 feet, about 4 feet to about 6 feet, about 4 feet to about 8 feet, about 4 feet to about 10 feet, about 4 feet to about 12 feet, about 4 feet to about 15 feet, about 5 feet to about 6 feet, about 5 feet to about 8 feet, about 5 feet to about 10 feet, about 5 feet to about 12 feet, about 5 feet to about 15 feet, about 6 feet to about 8 feet, about 6 feet to about 10 feet, about 6 feet to about 12 feet, about 6 feet to about 15 feet, about 8 feet to about 10 feet, about 8 feet to about 12 feet, about 8 feet to about 15 feet, about 10 feet to about 12 feet, about 10 feet to about 15 feet, or about 12 feet to about 15 feet. In some embodiments, the width of the shielded room or chamber is about 2 feet, about 4 feet, about 5 feet, about 6 feet, about 8 feet, about 10 feet, about 12 feet, or about 15 feet. In some embodiments, the width of the shielded room or chamber is at least about 2 feet, about 4 feet, about 5 feet, about 6 feet, about 8 feet, about 10 feet, or about 12 feet. In some embodiments, the width of the shielded room or chamber is at most about 4 feet, about 5 feet, about 6 feet, about 8 feet, about 10 feet, about 12 feet, or about 15 feet.

In some embodiments, the depth of the shielded room or chamber is about 2 feet to about 15 feet. In some embodiments, the depth of the shielded room or chamber is about 2 feet to about 4 feet, about 2 feet to about 5 feet, about 2 feet to about 6 feet, about 2 feet to about 8 feet, about 2 feet to about 10 feet, about 2 feet to about 12 feet, about 2 feet to about 15 feet, about 4 feet to about 5 feet, about 4 feet to about 6 feet, about 4 feet to about 8 feet, about 4 feet to about 10 feet, about 4 feet to about 12 feet, about 4 feet to about 15 feet, about 5 feet to about 6 feet, about 5 feet to about 8 feet, about 5 feet to about 10 feet, about 5 feet to about 12 feet, about 5 feet to about 15 feet, about 6 feet to about 8 feet, about 6 feet to about 10 feet, about 6 feet to about 12 feet, about 6 feet to about 15 feet, about 8 feet to about 10 feet, about 8 feet to about 12 feet, about 8 feet to about 15 feet, about 10 feet to about 12 feet, about 10 feet to about 15 feet, or about 12 feet to about 15 feet. In some embodiments, the depth of the shielded room or chamber is about 2 feet, about 4 feet, about 5 feet, about 6 feet, about 8 feet, about 10 feet, about 12 feet, or about 15 feet. In some embodiments, the depth of the shielded room or chamber is at least about 2 feet, about 4 feet, about 5 feet, about 6 feet, about 8 feet, about 10 feet, or about 12 feet. In some embodiments, the depth of the shielded room or chamber is at most about 4 feet, about 5 feet, about 6 feet, about 8 feet, about 10 feet, about 12 feet, or about 15 feet.

A shielded room or chamber can be of any shape. In some embodiments, a shielded room or chamber is in a substantially rectangular shape. In some embodiments, a shielded room or chamber is in a substantially cubic shape. In some embodiments, a shielded room or chamber is substantially cylindrical in shape. In some embodiments, a shielded room or chamber has tapered or conical elements. In some embodiments, a shielded room or chamber comprises an internal volume configured for placing an individual, a sensor, a mobility device, or a combination thereof within the internal volume.

In some embodiments, a shielded room or chamber has an internal volume. In some embodiments, the internal volume of the shielded room or chamber is about 12 cubic feet to about 2,000 cubic feet. In some embodiments, the internal volume of the shielded room or chamber is about 12 cubic feet to about 20 cubic feet, about 12 cubic feet to about 50 cubic feet, about 12 cubic feet to about 100 cubic feet, about 12 cubic feet to about 250 cubic feet, about 12 cubic feet to about 500 cubic feet, about 12 cubic feet to about 1,000 cubic feet, about 12 cubic feet to about 2,000 cubic feet, about 20 cubic feet to about 50 cubic feet, about 20 cubic feet to about 100 cubic feet, about 20 cubic feet to about 250 cubic feet, about 20 cubic feet to about 500 cubic feet, about 20 cubic feet to about 1,000 cubic feet, about 20 cubic feet to about 2,000 cubic feet, about 50 cubic feet to about 100 cubic feet, about 50 cubic feet to about 250 cubic feet, about 50 cubic feet to about 500 cubic feet, about 50 cubic feet to about 1,000 cubic feet, about 50 cubic feet to about 2,000 cubic feet, about 100 cubic feet to about 250 cubic feet, about 100 cubic feet to about 500 cubic feet, about 100 cubic feet to about 1,000 cubic feet, about 100 cubic feet to about 2,000 cubic feet, about 250 cubic feet to about 500 cubic feet, about 250 cubic feet to about 1,000 cubic feet, about 250 cubic feet to about 2,000 cubic feet, about 500 cubic feet to about 1,000 cubic feet, about 500 cubic feet to about 2,000 cubic feet, or about 1,000 cubic feet to about 2,000 cubic feet. In some embodiments, the internal volume of the shielded room or chamber is about 12 cubic feet, about 20 cubic feet, about 50 cubic feet, about 100 cubic feet, about 250 cubic feet, about 500 cubic feet, about 1,000 cubic feet, or about 2,000 cubic feet. In some embodiments, the internal volume of the shielded room or chamber is at least about 12 cubic feet, about 20 cubic feet, about 50 cubic feet, about 100 cubic feet, about 250 cubic feet, about 500 cubic feet, or about 1,000 cubic feet. In some embodiments, the internal volume of the shielded room or chamber is at most about 20 cubic feet, about 50 cubic feet, about 100 cubic feet, about 250 cubic feet, about 500 cubic feet, about 1,000 cubic feet, or about 2,000 cubic feet.

In some embodiments, a measurement collected from a sensor is collected from inside an internal volume of a shielded room or chamber. In some embodiments, a measurement is collected in the absence of an individual. In some embodiments, a measurement is collected in the presence of an individual. In some embodiments, a shield comprises a portion of an internal volume having a greater spatial homogeneity or greater amount of noise reduction as compared with a different portion. For example, a tapered end or a conical shaped end of an internal volume has greater spatial homogeneity of a measurement, a noise reduction, or both as compared to a cylindrical shaped end. In some embodiments, an individual is positioned within an internal volume of a shield such that an area of the subject desired to be measured by the sensor is positioned within a portion of the internal volume having greater spatial homogeneity of a measurement, a reduction in noise, or both.

In some embodiments, altering a height of a shielded room or chamber, altering a depth of a shielded room or chamber, altering a width of a shielded room or chamber, or altering a shape of a shielded room or chamber (such as a tapering) alters noise reduction and quality of a measurement within an internal volume of a shield. Each is independently altered or collectively altered to optimize noise reduction or improve quality of a measurement taken by a sensor.

In some embodiments, a shield comprises a coil, such as a Helmholtz coil. In some embodiments, a coil generates a current within the coil. In some embodiments, addition of a coil to a shield improves a quality of a measurement (such as a spatial homogeneity of a measurement), reduces a noise, or a combination thereof. In some embodiments, the presence of one or more coils creates homogenous regions within the shielded room or chamber. In some embodiments, the presence of one of more coils creates excitable regions within the shielded room or chamber. In some embodiments, a shield comprises a plurality of coils. A shield, in some embodiments, comprises a single coil. A shield, in some embodiments, comprises two coils. A shield, in some embodiments, comprises three coils. A shield, in some embodiments, comprises from 1 to 3 coils. In some embodiments, a coil is positioned within a portion of a shield. In some embodiments, a coil is positioned within a portion of a shield that a measurement occurs. In some embodiments, a position of a coil is adjustable, such as by a controller or by a user. In some embodiments, a position of a coil is adjusted for each measurement of a sensor. In some embodiments, a position of a coil is pre-programed accordingly to a type of measurement of a sensor. In some embodiments, a position of a coil is adjustable with an accuracy of from about 0.1 inches to about 5 inches. In some embodiments, a coil provides feedback to a user or to a controller that a desired positioned is achieved by the coil. In some embodiments, a feedback from a coil to a user or to a controller occurs prior to a measurement, during a measurement, or after a measurement of a sensor. In some embodiments, a feedback from a coil confirms that a desired position (such as a position corresponding to a position of an individual desired to be measured) is reached.

In some embodiments, a shield is modular. In some embodiments, a shield or portion thereof is disposable. In some embodiments, a shielded room or chamber is configured to accept a whole individual. In some embodiments, a shielded room or chamber is configured to accept a whole individual and one or more mobility devices. In some embodiments, a shielded room or chamber is configured to accept at least a portion of an individual, at least a portion of a sensor array, or a combination thereof. A portion of an individual, in some embodiments, comprises a head, an arm, or a leg that is placed into an inner volume of a shield. A portion of an individual, in some embodiments, comprises an individual from a mid-section to a head or from a mid-section to a foot. In some embodiments, a shield is not modular. In some embodiments, a shield is configured to interact with one or more modular units. For example, a modular unit, such as base unit, is modular and configured to modulate in relation to a shield that is stationary or non-modular.

In some embodiments, a shielded room or chamber or portion thereof is configured for subject comfort. In some embodiments, a shielded room or chamber or portion thereof is configured with lighting, such as an internal volume of a shielded room or chamber, in some embodiments, comprises a lighting source. In some embodiments, a shielded room or chamber or portion thereof is configured with venting, such as one or more ports or openings, such as one or more openings positioned on an internal surface of a shielded room or chamber. In some embodiments, the shielded room or chamber or a portion thereof comprises a projector. In some embodiments, a projector allows information to be displayed to a subject inside of the shielded room or chamber. In some embodiments, the projector may display a television show or movie to increase the comfort of an individual in the shielded room or chamber. In some embodiments, the shielded room or chamber may comprise a speaker. In some embodiments, the speakers allow information or directions to be articulated to a subject inside the shielded room or chamber. In some embodiments, the speakers may play music or other audio programming to increase the comfort of an individual within the shielded room or chamber. In some embodiments, the shielded room or chamber comprises a camera. The camera may allow a subject to be visually monitored while inside the shielded room or chamber. In some embodiments, the shielded room or chamber comprises a microphone. The microphone may allow the subject to be audibly monitored.

A shield, in some embodiments, comprises a single material. A shield, in some embodiments, comprises more than one material. A shield or a portion thereof, in some embodiments, comprises a metal, a metal alloy, or a combination thereof. A shield or a portion thereof, in some embodiments, comprises a permalloy or a mumetal (or “mu-metal”). A shield or a portion thereof, in some embodiments, comprises aluminum, copper, gold, iron, nickel, platinum, silver, tin, zinc, or any combination thereof. A shield or a portion thereof, in some embodiments, comprises brass, bronze, steel, chromoly, stainless steel, titanium, or any combination thereof.

A shield or a portion thereof, in some embodiments, comprises nickel, iron, or a combination thereof. In some embodiments, a shield or portion thereof comprises from about 70% to about 90% by weight of nickel. In some embodiments, a shield or portion thereof comprises from about 75% to about 85% by weight of nickel. In some embodiments, a shield or portion thereof comprises from about 10% to about 30% by weight of iron. In some embodiments, a shield or portion thereof comprises from about 15% to about 25% by weight of iron. In some embodiments, a shield or portion thereof comprises from about 70% to about 90% by weight of nickel and from about 10% to about 30% by weight of iron. In some embodiments, a shield or portion thereof comprises from about 40% to about 60% by weight nickel and about 50% to about 60% by weight of iron. In some embodiments, a shield or portion thereof comprising a permalloy or a mumetal also comprises one or more additional elements such as molybdenum.

A shield or portion thereof, in some embodiments, comprises a material having a high permeability. For example, a material, in some embodiments, comprises a relative permeability of from about 50,000 to about 900,000 as compared to for example steel having a relative permeability of from about 4,000 to about 12,000. A material, in some embodiments, comprises a relative permeability of from about 75,000 to about 125,000. A material, in some embodiments, comprises a relative permeability of from about 400,000 to about 800,000. A material, in some embodiments, comprises a relative permeability of greater than about 50,000. A material, in some embodiments, comprises a relative permeability of greater than about 75,000. A material, in some embodiments, comprises a relative permeability of greater than about 100,000. A material, in some embodiments, comprises a relative permeability of greater than about 200,000. A material, in some embodiments, comprises a relative permeability of greater than about 300,000. A material, in some embodiments, comprises a relative permeability of greater than about 400,000. A material, in some embodiments, comprises a relative permeability of greater than about 500,000. A material, in some embodiments, comprises a relative permeability of greater than about 600,000. A material, in some embodiments, comprises a relative permeability from about 80,000 to about 900,000. A material, in some embodiments, comprises a relative permeability from about 400,000 to about 800,000.

In some embodiments, a shield is monolith in form. In some embodiments, a shield is formed of a plurality of subcomponents configured together. In some embodiments, a shield is 3D printed. A shield, in some embodiments, comprises a material formed in a hydrogen furnace, such as a shield comprising one or more materials annealed in a hydrogen furnace.

In some embodiments, a shield or shielded room or chamber comprises a shielded door. In some embodiments, the door of the shielded room or chamber is comprised of identical material as the rest of the shielded room or chamber. In some embodiments, the shielded room or chamber has identical metal shielding as the rest of the shielded room or chamber. In some embodiments, the material of the door is configured to enhance the magnetic performance and homogeneity inside the shielded room or chamber. In some embodiments, the spacing between the door and the rest of the shielded room or chamber is configured to increase magnetic performance and homogeneity inside the shielded room or chamber.

Described herein are devices and systems configured to sense a magnetic field associated with, for example, a tissue, a body part, or an organ of an individual. In some embodiments of the devices and systems described herein, a device for sensing a magnetic field comprises a magnetically shielded room or chamber and one or more magnetic field sensors.

In some embodiments of the devices and systems described herein, a device for sensing a magnetic field comprises one or more magnetic field sensors such as, for example, one or more OPMs.

In some embodiments, the device for sensing a magnetic field comprises a shielded room or chamber that is modular. In some embodiments, the module (herein referred to interchangeably with “modular component”) used is application specific. In some embodiments, there are module specific positioning procedures for different applications.

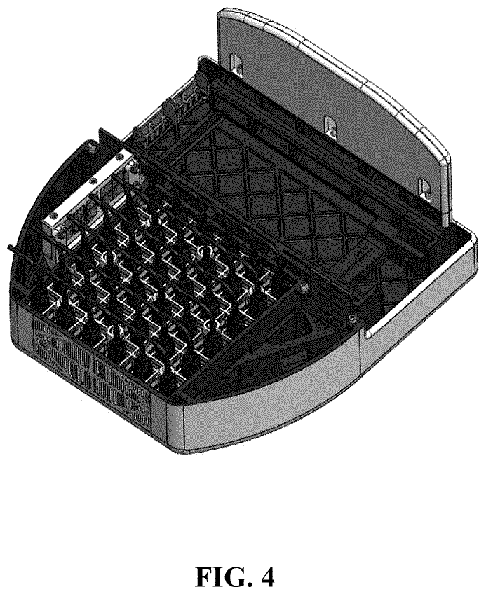

In some embodiments, the shielded room or chamber comprises a module for cardiac applications. In some embodiments, a module for cardiac applications comprises a MCG module. In some embodiments, the module for cardiac applications comprises sensors that are fixed in location. In some embodiments, a patient sits or stands in the shielded room for the module for cardiac applications. In some embodiments, a patient presses themselves against the sensor module with reference to a specific anatomical landmark coinciding with the sensor array. FIG. 4 shows an example of a sensor array (sensors shown in black, cables cut for clarity). For precise positioning of this sensor array above the patient's heart, the housing can be raised, lowered and translated in a transverse direction (shoulder to shoulder) via a manually operated gear mechanism.

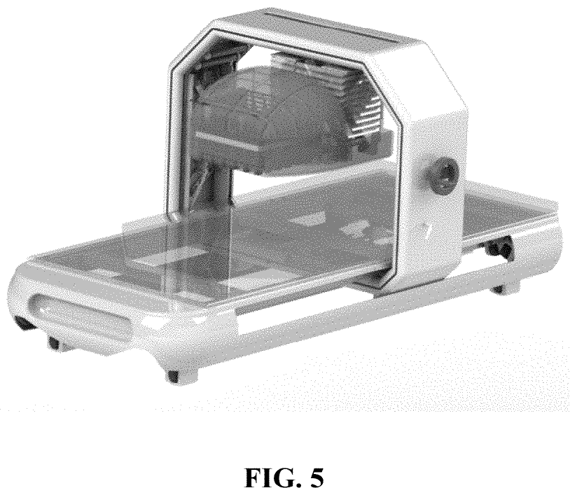

In some embodiments, the shielded room or chamber comprises a module for neurological applications. In some embodiments, the module for neurological applications comprises a MEG module. In some embodiments, a “helmet” or “head cage” comprising sensors can be maneuvered to the patient and positioned on a patient's head with reference to specific anatomical landmarks coinciding with a location on the sensor array. In some embodiments, the neurological module measures neuron action potential activity. In some embodiments, the neurological module measures magnetic fields produced by a patient's electrical currents present in the brain. In some embodiments, the neurological module performs magnetoencephalography. In some embodiments, the neurological module is used to identify the source or location within the brain of an epileptic seizure. FIG. 5 shows an example of a 3D rendering of a sensor head cage mounted on a bed of a shield (the patient's head would be on the left, chest underneath the arch within the shield).

In some embodiments, the shielded room or chamber comprises a module for magnetorelaxometry. In some embodiments, a movable coil (i.e., magnetization coil) and magnetometer setup is used for site specific measurements of magnetorelaxometry measurements. In some embodiments, the site specific measurements are operator positioned. In some embodiments, the movable coil excites tissue while sensors pick up relaxation curves, as applicable. In some embodiments, the magnetorelaxometry module measures Neel and Brownian relaxation of particles in the body after being exposed to an external magnetic stimulus. In some embodiments, the particles do not originate from inside the body. In some embodiments, the particles are injected into a patient. In some embodiments, the particles are naturally occurring within a patient.

In some embodiments, the shielded room or chamber comprises a module for ultra-low field MRI. In some embodiments, the ultra-low field MM module utilizes inbuilt coils in the room for low field generation. In some embodiments, the ultra-low field MM module utilizes inbuilt coils in the sensor array to pick up magnetic signal. The inbuilt coils may be located in the MM region to pick up magnetic signal. Thus, the coil may be used to produce the image of a volume (e.g., an individual).

In some embodiments, one module is used for a patient. In some embodiments, two or more modules are used on a single patient. For example, a patient may enter the shielded room or chamber and be subject to a MCG module and MEG module. Therefore, one shielded room or chamber may be used for multiple applications, and a patient does not have to relocate to another testing area or device.

In some embodiments, the shielded room or chamber comprises a modular mounting system. In some embodiments, the mounting system is a common rail mounting system. In some embodiments, the mounting system is flush with the walls of the room or chamber. In some embodiments, the mounting system comprises aluminum studs or prefabricated pegboard, or a combination thereof, for modular mounting. In some embodiments, the mounting system may comprise one or more modules. In some embodiments, the mounting system comprises a module for cardiac applications, a module for neurological applications, a module for magnetorelaxometry, or a module for ultra-low field MRI, or a combination thereof. In some embodiments, the shielded room or chamber comprises a Gantry system. In some embodiments, the Gantry system comprises a module for cardiac applications, a module for neurological applications, a module for magnetorelaxometry, or a module for ultra-low field MRI, or a combination thereof. In some embodiments, the shielded room or chamber comprises a Gantry system.

In some embodiments of the devices and systems described herein, a device for sensing a magnetic field comprises one or more coupling mechanisms for receiving and coupling with one or more sensors. In some embodiments of the systems and devices described herein, a device for sensing a magnetic field coupler comprises one or more arms or extensions that connect with the mobile base unit. In some embodiments of the devices and systems described herein, a device for sensing a magnetic field includes one or more extensions or arms configured to move, rotate, and/or articulate so as to position one or more sensors for sensing a magnetic field within proximity to an individual whose magnetic field is to be sensed.

In some embodiments, a device or system as described herein comprises a mechanical housing that comprises one or more nonferrous materials, such as, for example, an aluminum alloy, a rubber, a plastic, a wood or any combination thereof to minimize an amount of interference seen in a biomagnetic signal from a device or system itself.

### Embodiments

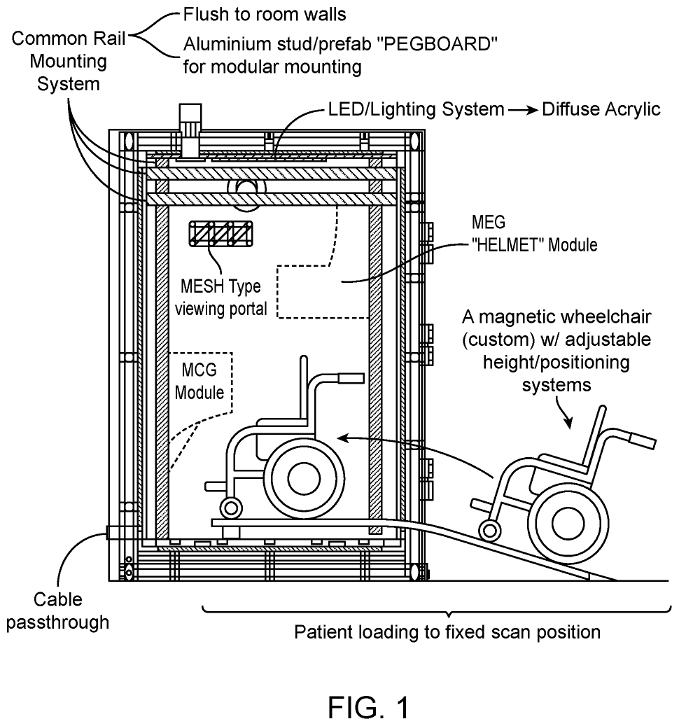

FIG. 1 shows a magnetically shielded room or chamber. The shielded room or chamber may comprise a common rail mounting system. The rail mounting system may be a magnetic rail system. In some embodiments, the common rail mounting system may be used to mount one or more modules. The mounting system may be flush against the walls of the shielded room or chamber. In some embodiments, the mounting system comprises aluminum studs, or prefabricated pegboard, or a combination thereof. The mounting system may be used for modular mounting. The modules may be removed or repositioned on the mounting system. New modules may be added to an existing mounting system.

In FIG. 1, the shielded chamber comprises a magnetocardiography (“MCG”) module. In some embodiments, a MCG is a visual representation of the magnetic fields produced by the electrical activity of the heart. In some embodiments, the MCG module comprises a fixed location of sensors. In some embodiments, the MCG module can be used while the patient is seated, standing, or lying down, or a combination thereof. In some embodiments, when using the MCG module, a patient presses themselves against the sensor module with reference to specific anatomical landmark coinciding with the sensor array. In some embodiments, the magnetocardiography module is a fetal magnetocardiography module.

In FIG. 1, the shielded chamber also comprises a magnetoencephalography (“MEG”) module. In some embodiments, the MEG module measures magnetic fields produced by a patient's electrical currents present in the brain. In some embodiments, the MEG module is used to identify the source or location within the brain of an epileptic seizure. In some embodiments, as shown in FIG. 1, the MEG module comprises a “helmet” or “head cage” comprising sensors which can be maneuvered to the patient and positioned on their head with reference to specific anatomical landmarks coinciding with a location on the sensor array. In some embodiments, the “helmet” of sensors may be repositioned to be in close proximity to a patient's head. In some embodiments, the MEG module can be used while the patient is seated, standing, or lying down, or a combination thereof. In some embodiments, the magnetocardiography module is a fetal magnetoencephalography module.

In some embodiments, the shielded chamber has one or more windows. In some embodiments, the one or more windows allows the inside of the chamber to be viewed from outside of the chamber. In some embodiments, one or more windows is made of mesh.

In some embodiments, the shielded room or chamber has a lighting system. In some embodiments, the lighting system comprises one or more light-emitting diodes (“LEDs”). In some embodiments, the LEDs comprise an acrylic light diffuser.

A patient may be in a wheelchair. In some embodiments, the patient is wheeled into the shielded room or chamber through a door. In some embodiments, the shielded room or chamber comprises a ramp that allows a patient to be wheeled into the shielded chamber or room. A patient may enter the shielded room or chamber through a door. In some embodiments, a patient enters the shielded room or chamber with the aid of a wheelchair. The wheelchair may be made of material that is not magnetic. The wheelchair may be adjustable. For example, the wheelchair may comprise an adjustable height system or an adjustable positioning system, or a combination thereof. In some embodiments, the wheelchair can be reclined. In some embodiments, the patient remains sitting in the wheelchair during device use.

In another example, a patient may be wheeled into the shielded room or chamber on a hospital bed. The patient may remain lying in the hospital bed during device use. In some embodiments, a patient is positioned or loaded outside of the shielded room or chamber. In some embodiments, a patient is positioned or loaded inside of the shielded room or chamber. In some embodiments, a mobility device, like a wheelchair or hospital bed, may be adjusted or repositioned prior to device use. In some embodiments, a mobility device, like a wheelchair or hospital bed, may be adjusted or repositioned during device use. In some embodiments, a patient be repositioned within the shielded room or chamber for application of a different module. For example, a patient may initially be positioned in a seated position near the MCG module and the wheelchair may be readjusted to a higher position for the MEG module. In some embodiments, the modules may change positions within the shielded room or chamber. In some embodiments, the patient remains in a fixed position during device use, and the modules are positioned accordingly around the patient.

The shielded room or chamber may have a viewing area to enable patient monitoring and/or improve patient comfort. The viewing area may be implemented using at least one passthrough, window or hole inserted within at least one of the walls of the shielded room. Additionally, the shielded room or chamber may comprise a projector and/or speaker to provide external stimulus for experiments or provide entertainment for the patient.

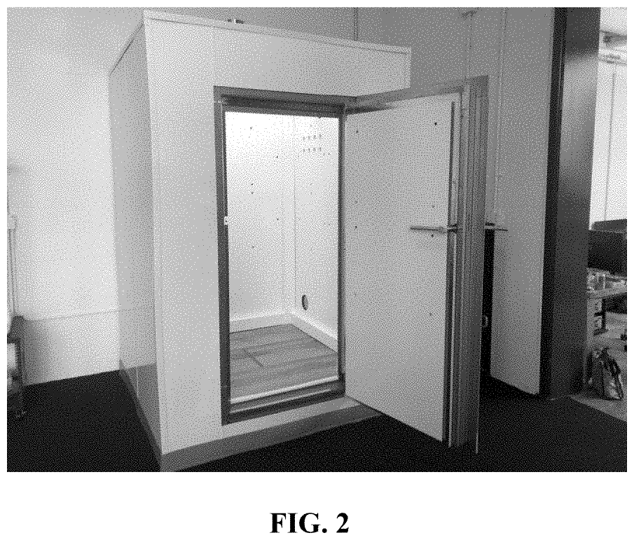

FIG. 2 shows a photograph of a magnetically shielded chamber or room with the shielded door opened.

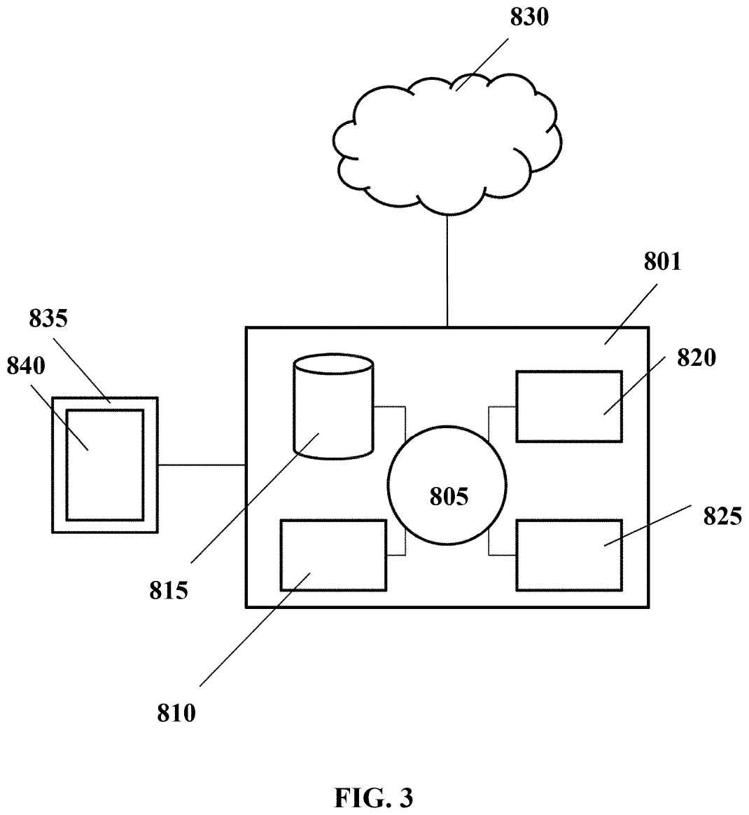

FIG. 3 shows a computer system 801 that is programmed or otherwise configured to direct operation of a device or system as described herein, including movement of a base unit, movement of a shield, movement of a mobile cart, movement of a sensor array, acquisition of a measurement, comparison of a measurement to a reference measurement, or any combination thereof. The computer system 801 regulates various aspects of (a) movement of one or more device or system components, (b) operation of one or more sensors, (c) adjustment of one or more parameters of a sensor, (d) computationally evaluation of one or more measurements of a device or system, (e) display of various parameters including input parameters, results of a measurement, or any combination of any of these. In some embodiments, a computer system 801 is an electronic device of a user (e.g. smartphone, laptop) or, in some embodiments, is remotely located with respect to the electronic device. The electronic device, in some embodiments, is a mobile electronic device.

The computer system 801 includes a central processing unit (CPU, also “processor” and “computer processor” herein) 805, which, in some embodiments, is a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 is configured as a data storage unit (or data repository) for storing data. The computer system 801 is operatively coupled to a computer network (“network”) 830 with the aid of the communication interface 820. The network 830 is the Internet, an internet and/or extranet, or an intranet and/or extranet that is in communication with the Internet. The network 830 in some embodiments is a telecommunication and/or data network. The network 830 includes one or more computer servers, which enable distributed computing, such as cloud computing. The network 830, in some embodiments, with the aid of the computer system 801, implements a peer-to-peer network, which enables devices coupled to the computer system 801 to behave as a client or a server.

The CPU 805 is configured to execute a sequence of machine-readable instructions, which are be embodied in a program or software. The instructions are stored in a memory location, such as the memory 810. The instructions are directed to the CPU 805, which is subsequently program or otherwise configure the CPU 805 to implement methods of the present disclosure. Examples of operations performed by the CPU 805 include fetch, decode, execute, and writeback.

The CPU 805 is part of a circuit, such as an integrated circuit. One or more other components of the system 801 are included in the circuit. In some embodiments, the circuit is an application specific integrated circuit (ASIC).

The storage unit 815 stores files, such as drivers, libraries and saved programs. The storage unit 815 stores user data, e.g., user preferences and user programs. The computer system 801 in some embodiments include one or more additional data storage units that are external to the computer system 801, such as located on a remote server that is in communication with the computer system 801 through an intranet or the Internet.

The computer system 801 communicates with one or more remote computer systems through the network 830. For instance, the computer system 801 communicates with a remote computer system of a user (e.g., a second computer system, a server, a smart phone, an iPad, or any combination thereof). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user accesses the computer system 801 via the network 830.

Methods as described herein are implemented by way of machine (e.g., computer processor) executable code stored on an electronic storage location of the computer system 801, such as, for example, on the memory 810 or electronic storage unit 815. The machine executable or machine readable code is provided in the form of software. During use, the code is executed by the processor 805. In some embodiments, the code is retrieved from the storage unit 815 and stored on the memory 810 for ready access by the processor 805. In some situations, the electronic storage unit 815 is precluded, and machine-executable instructions are stored on memory 810.

A machine readable medium, such as computer-executable code, takes many forms, including but not limited to, a tangible storage medium, a carrier wave medium or physical transmission medium. Non-volatile storage media include, for example, optical or magnetic disks, such as any of the storage devices in any computer(s) or the like, such as is used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media takes the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer reads programming code and/or data. Many of these forms of computer readable media is involved in carrying one or more sequences of one or more instructions to a processor for execution.

The computer system 801, in some embodiments, includes or is in communication with an electronic display 835 that comprises a user interface (UI) 840 for providing, for example, a graphical representation of one or more signals measured, one or more reference signals, one or more parameters that is input or adjusted by a user or by a controller, or any combination thereof. Examples of UI's include, without limitation, a graphical user interface (GUI) and web-based user interface.

Methods and systems of the present disclosure are, in some embodiments, implemented by way of one or more algorithms. An algorithm, in some embodiments, is implemented by way of software upon execution by the central processing unit 805. The algorithm is, for example, comparing a signal to a reference signal.

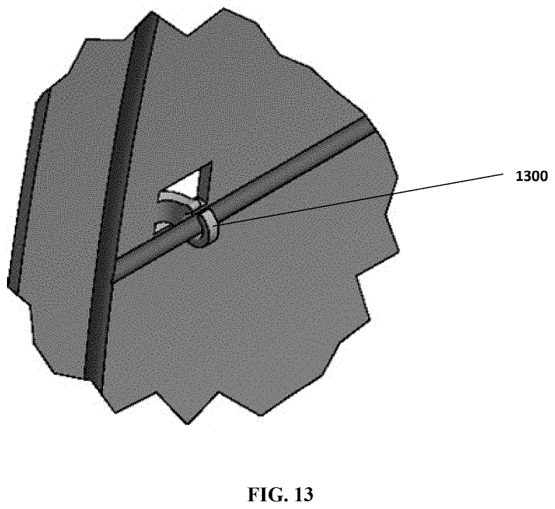

FIG. 13, shows an exemplary hook 1300 configured to span a portion of or an entire volume of a shield. In some embodiments, one or more hooks 1300 are operatively connected to a wiring (such as holding a wiring) and is designed to transmit analog electrical signals, digital electrical signals, or a combination thereof. In some embodiments, one or more hooks 1300 are positioned along a single plane of a shield. In some embodiments, hooks 1300 are positioned along more than one plane of a shield. Hooks are positioned along multiple planes. In some embodiments, hooks 1300 are positioned on an inside surface of a shield. In some embodiments, hooks 1300 are positioned circumferentially about a shield at a single cross section. In some embodiments, hooks 1300 are positioned circumferentially about a shield and continuing along a length of a shield. In some embodiments, hooks 1300 are configured to hold an electrical coil system, such as an electrical coil system designed to eliminate an accumulated magnetic field. In some embodiments, hooks 1300 are configured to hold an electrical coil system, such as an electrical coil system designed to create a homogenous magnetic environment inside a shield. In some embodiments, an electrical coil system is configured to employ the use of a wire of variable gauge. An exemplary wire gauge suitable for use with devices and systems described herein is 28 AWG shown in FIG. 13.

In some embodiments, performance of a magnetometer is improved with equilibration. In these embodiments, a gradient of 1 nT/m is achieved within the shield. Equilibration, in some embodiments, comprises the process of degaussing.

In some embodiments, a shield configured for utilization of the equilibration process comprises an arrangement of coils. Typically the coils are arranged in one or more layers. In some embodiments, a shield comprises an inner coil layer and one or more outer coil layers, inner coils for an innermost layer and outer coils for each of the outer layers.

In some embodiments, the inner coils are (for 90 cm diameter of the cylinders) distributed in 45 degrees to effectively form 8 coils. The mechanical mounting precision is about +/−2 cm per wire. Many different configurations are acceptable for the outer coils generally. In some embodiments, a shield comprises 1 outer coil. In some embodiments, a shield comprises 2 outer coils. In some embodiments, a shield comprises 3 outer coils. In some embodiments, a shield comprises 4 outer coils. In some embodiments, a shield comprises 5 outer coils. In some embodiments, a shield comprises 6 outer coils. In some embodiments, a shield comprises 7 outer coils. In some embodiments, a shield comprises 8 outer coils. In some embodiments, a shield comprises 9 outer coils. In some embodiments, a shield comprises 10 outer coils.

In some embodiments, at least the inner layer must be electrically isolated. In some embodiments, ESD PVC is used instead of regular plastic just to avoid charge up effects, which disturb the magnetometers.

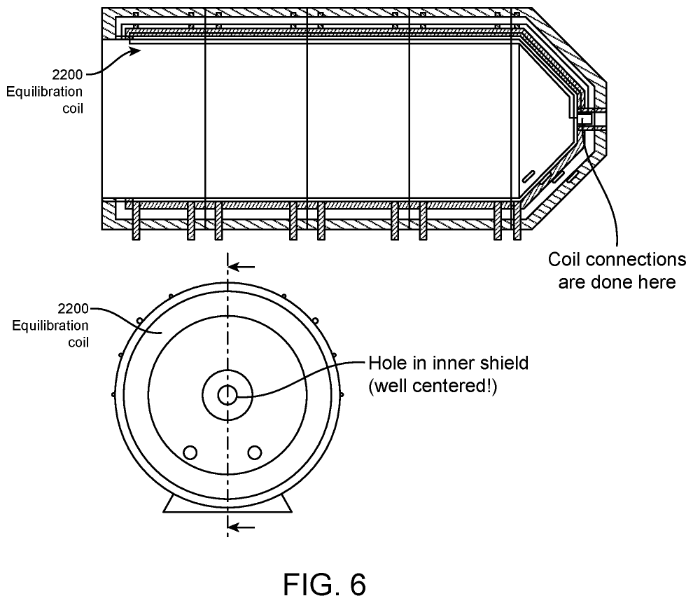

FIGS. 6-8 illustrate components within an embodiment of a shield. The coil layouts and equilibration function may approximate those associated with shields used with the present disclosure.

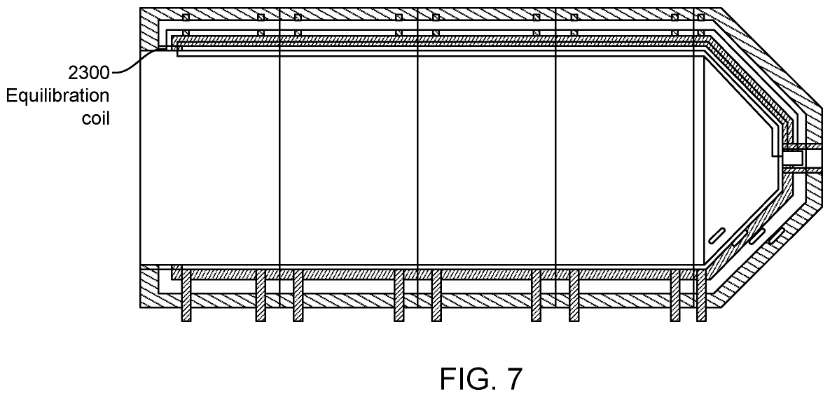

FIG. 6 shows an exemplary layout of one inner coil 2200 positioned in an embodiment of a shield. FIG. 7 shows an exemplary layout of outer coils 2300 positioned in an embodiment of a shield.

In some embodiments, a connection to an amplifier (or transformer) is opened during the measurements with the magnetic field probes. In some embodiments, this is achieved using a mechanical relay.

The wire dimensions may typically at least 2.5 mm² In some embodiments, a device may have three turns per eighth of the coil, resulting in 24 turns. The permeability may be between about 160,000 to 380,000 henry/meter. So the 24 turns would be about 1 Ohm and with 10 A saturation current, this gives 10 V.

In some embodiments, an equilibration sequence would be a 30 s sequence with linearly decreasing envelope, starting from saturation of the inner layer. This sequence may be performed each time a large change in the field is applied. During regular operation, the sequence may be performed one to three times daily. The outer shields must be equilibrated only once, when the shield is installed, or when the external fields change direction by 90 degrees or so, using the same amplifier. (Therefore, there must be a similar amount of turns for the coils, to use the same equipment).

In some embodiments, the coils for equilibration are individual wires with gold plated contacts. Due to magnetization issues, no Ni substrates or coatings can be used for connectors inside. In some embodiments, the required level of precision the equilibration coils of the outer shield can be placed randomly without special precautions, whereas the inner coils require at least a 6 fold symmetry for the distribution of the current to obtain a reasonably shaped residual field for 60 cm diameter and 8 for 1 m diameter. For the demonstrated project we chose connectors from brass with gold coating without a nickel intermediate layer to avoid excessive magnetization. All connectors must be placed outside the inner shield layer. Their magnetization (on this level) is not relevant for the residual field inside.

An equilibration process employed in some embodiments of the shields described herein, is a process for bringing magnetizable material in an equilibrium with a surrounding magnetic field. In some embodiments, this is done by applying a sinusoidal current around a magnetizable material. The oscillation is extremely well centered around zero and is large enough to saturate the material in both directions. By decreasing the amplitude to zero, a very low magnetic field strength outside the magnetizable material (inside the cylinder) is obtained. For initial tests, a linearly decreasing envelope is useful, as it is a very reliable function. This model is programmed into the equilibration unit. An exponentially decreasing function may be advantageous in future. The pre-set function (which can be changed by the user on the PC) is shown below:

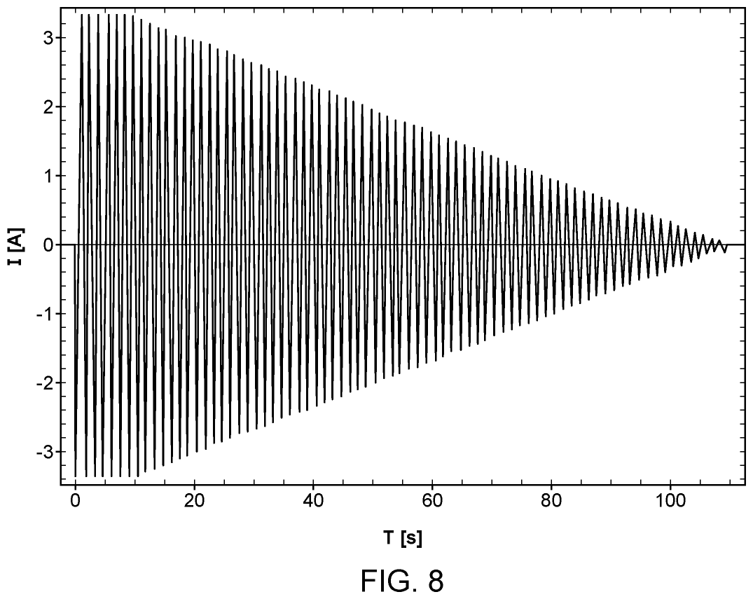

FIG. 8 shows a typical equilibration function. At the beginning, the maximum current is kept for 10 cycles and then decreased until zero amplitude is reached. Note that at the ultimate performance level, many options for changing and improvements are available.

In some embodiments, the equilibration coils are connected to the electrical equipment using twisted-pair cables. No RF shielding or other precautions are required, as higher frequencies are damped by the inductance of the shielding material and coil configuration (mH range).

In some embodiments, a computing device programs a sinusoidal function with envelope function, which is converted to a voltage signal by an NI 6281 data acquisition device. The voltage is fed to a voltage divider and then drives a power amplifier. The function can be set by the user and is programmable. The timing resolution of the curve is 10 kHz.

In some embodiments, inside the control box there is a box with potentiometers. These potentiometers can be adjusted manually to set the ratio of DAC voltage to current out of the amplifier. This minimizes any bit-size effects for the residual field (16 bit for 20 V=0.3 mV resolution). From experience, the optimization of this will be relevant for <0.5 nT residual fields. There are 2 potentiometers to tune different currents, they can then be selected via software. In case of a noisy environment, the voltage divider box is a useful place to add additional frequency filtering by a capacitor. In some embodiments, the band pass filtering of the amplifier will be sufficient for most applications.

In some embodiments, a power amplifier comprises a 4-quadrant amplifier which can be operated with large inductive loads and is intrinsically fail-safe against mistakes operation, e.g shortcuts, many inductive spikes etc. For magnetic equilibration, the amplifier should be used in current-controlled mode, but can also be operated in any configuration. Due to extreme noise requirements, it is preferable to change the coils around the magnetizable material (cross section and number of turns) to match the maximum power of the amplifier. The power is chosen to be very small to achieve extremely low noise operation. Band-pass filters can be set manually on the front side to reduce noise effects. The amplifier can be fully remote controlled via a sub-d connector on the back side. A unique feature if this amplifier is the possibility to adjust the base-line by 1% via an analog +/−10 V input, independent of the signal input.

In some embodiments, to perform DC measurements, the noise and the drift of the magnetic field probes is relevant. In some embodiments, one or more multiaxis Fluxgate magnetometers with <6 pT noise-amplitude (peak-to-peak) are employed. In some embodiments, a readout of, for example, one or more fluxgates is done with an analog input unit (e.g., a NI 6281, 18-bit) to provide sufficient resolution of the fluxgate analog signals (+/−10 V). USB control is used for data transfer to the PC, the NI unit is independently grounded and has an independent power supply. In some embodiments, the readout rate is set up to 625.000 samples per second.

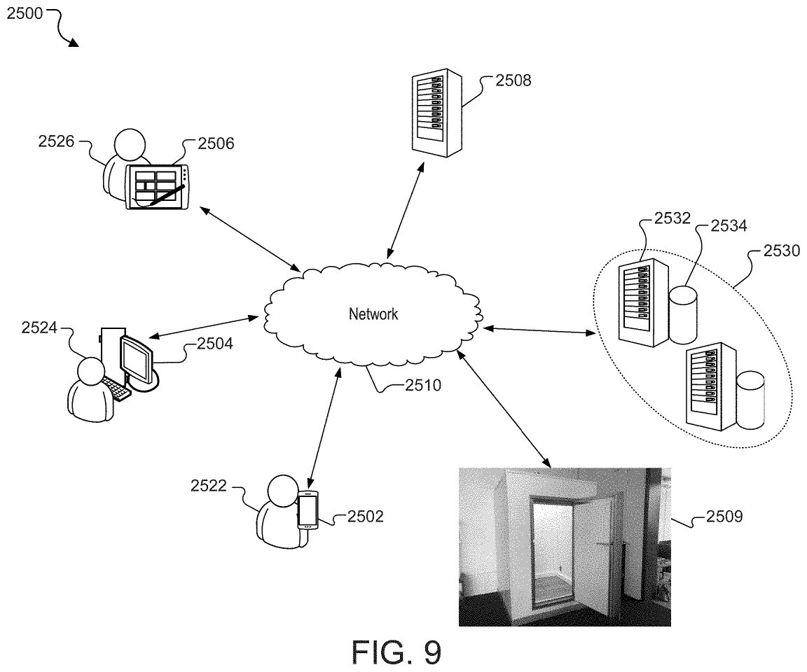

FIG. 9 depicts an example environment that can be employed to execute implementations of one or more embodiments of the platform 2500 of the present disclosure. The example platform 2500 includes computing devices 2502, 2504, 2506, 2508, medical device or system 2509, a back-end system 2530, and a network 2510. In some embodiments, the medical device or system 2509 comprises a shielded chamber or room where patient scans are taken. In some embodiments, the network 2510 includes a local area network (LAN), wide area network (WAN), the Internet, or a combination thereof, and connects web sites, devices (e.g., the computing devices 2502, 2504, 2506, 2508 and the medical device or system 2509) and back-end systems (e.g., the back-end system 2530). In some embodiments, the network 2510 can be accessed over a wired and/or a wireless communications link. For example, mobile computing devices (e.g., the smartphone device 2502 and the tablet device 2506), can use a cellular network to access the network 2510. In some embodiments, the users 2522-2526 includes physicians, patients, network technicians including network administrators and authorized programmers, nurses, residents, hospital administrators, insurers, and any other healthcare provider.

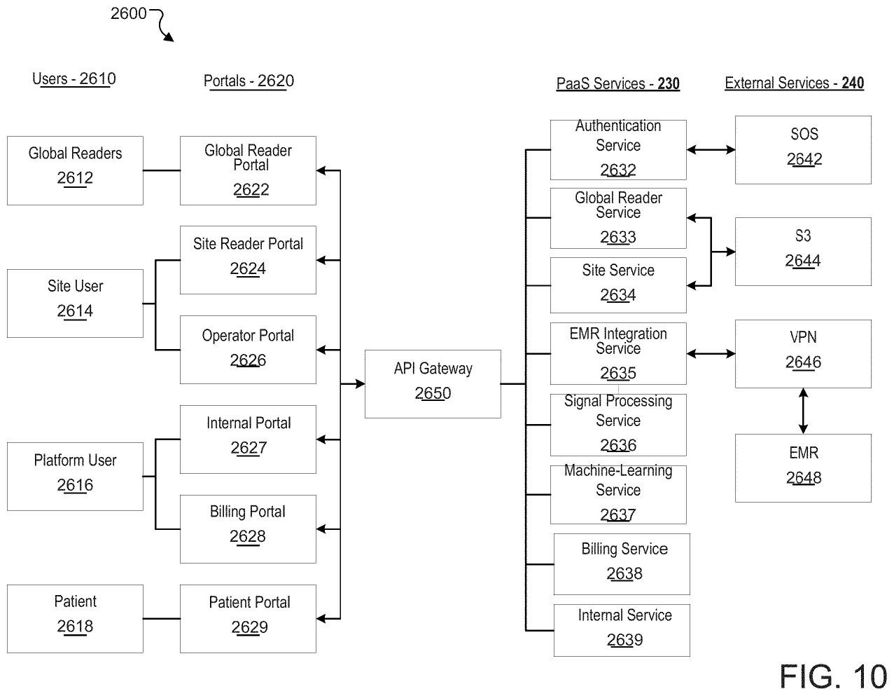

In the depicted example, the back-end system 2530 includes at least one server system 2532 and a data store 2534. In some embodiments, the at least one server system 2532 hosts one or more computer-implemented services and portals employed within the described platform, such as described in FIG. 10, that users 2522-2526 can interact with using the respective computing devices 2502-2506. For example, the computing devices 2502-2506 may be used by respective users 2522-2526 to generate and retrieve reports regarding patient scans taken by the medical device or system 2509 through services hosted by the back-end system 2530 (see FIG. 10). In some embodiments, the back-end system 2530 provides an API service with which the server computing device 2508 may communicate.

In some embodiments, back-end system 2530 includes server-class hardware type devices. In some embodiments, back-end system 2530 includes computer systems using clustered computers and components to act as a single pool of seamless resources when accessed through the network 2510. For example, such embodiments may be used in data center, cloud computing, storage area network (SAN), and network attached storage (NAS) applications. In some embodiments, back-end system 2530 is deployed using a virtual machine(s).

In some embodiments, the computing devices 2502, 2504, 2506 include any appropriate type of computing device, such as a desktop computer, a laptop computer, a handheld computer, a tablet computer, a personal digital assistant (PDA), a cellular telephone, a network appliance, a camera, a smart phone, an enhanced general packet radio service (EGPRS) mobile phone, a media player, a navigation device, an email device, a game console, or an appropriate combination of any two or more of these devices or other data processing devices. In the depicted example, the computing device 2502 is a smartphone, the computing device 2504 is a desktop computing device, and the computing device 2506 is a tablet-computing device. In some embodiments, the server computing device 2508 includes any appropriate type of computing device, such as described above for computing devices 2502-2506 as well as computing devices with server-class hardware. In some embodiments, the server computing device 2508 includes computer systems using clustered computers and components to act as a single pool of seamless resources. It is contemplated, however, that implementations of the present disclosure can be realized with any of the appropriate computing devices, such as those mentioned previously.

In some embodiments, the medical device or system 2509 comprises an array, such as a sensor array and a shield. In some embodiments, the medical device or system 2509 comprises a base unit and an array, such as a sensor array. In some embodiments, the medical device or system 2509 senses an electromagnetic field associated with one or more tissues or one or more organs of an individual. In some embodiments of the devices 2509, sensed electromagnetic field data associated with a heart is used to generate a magnetocardiogram. In these embodiments, the devices 2509 comprise a magnetocardiograph which may, for example, be a passive, noninvasive bioelectric measurement tool intended to detect, record, and display magnetic fields that are naturally generated by electrical activity of a heart. It should be understood that in some embodiments, an EMF that is sensed is associated with a brain of an individual and/or component of a nervous system of an individual (including both central and peripheral nervous systems). In some embodiments, an EMF that is sensed is associated with an organ of an individual, and/or a tissue of an individual, and/or a portion of a body of an individual, and/or an entire body of an individual.

In some embodiments, the medical device or system 2509 comprises at least one sensor, such as an optically pumped magnetometer (OPM) as a measurement tool, which may use nonradioactive self-contained alkali metal cells coupled with a closed pumping laser and photodetector setup to measure minute magnetic fields. In some embodiments, medical device or system 2509 comprises an array of two or more sensors. In some embodiments comprising an array, the two or more sensors of the array are the same type of EMF sensor, and, in some embodiments, an array of sensors comprises at least two different sensors. Non-limiting examples of EMF sensors suitable for use with the exemplary medical device or system 2509 include optically pumped magnetometer sensors, magnetic induction sensors, magneto-resistive sensors, SQUID sensors, fluxgate magnetometers, induction coil magnetometers, nitrogen vacancy diamonds, or magneto resistive sensors. In some embodiments, a fluxgate magnetometer is a Yttrium Iron Garnet (YIG) film fluxgate magnetometer.

In some embodiments, the medical device or system 2509 is configured to be used for cardiac applications, such as generating an MCG. In other embodiments, the medical device or system 2509 is used to sense an EMF associated with different parts of the body or for various diseases or conditions.

In some cases, the medical device or system 2509 is employed for a prognostic method, such as predicting a likelihood of a subject developing a disease or condition; a diagnostic method, such as confirming a diagnosis or providing a diagnosis to a subject for a disease or condition; or a monitoring method, such as monitoring a progression of a disease or condition in a subject, monitoring an effectiveness of a therapy provided to a subject, or a combination thereof.

In some embodiments, the medical device or system 2509 uses one or more OPMs in an n×n array (or grid) or alternative geometric configuration to collect magnetic field data at n discrete locations over a portion of a body of an individual (such as a chest area), which in some embodiments is digitized using pickup electronics and in some embodiments is connected to a computer for recording and displaying this data. It should be understood, however, that the medical device or system 2509 is suitable for measuring an electromagnetic field associated with any type of tissue, for example, utilizing OPMs.

In some embodiments, the medical device or system 2509 is configured to sense an EMF associated with, for example, a tissue, a body part, or an organ of an individual. In some embodiments, the medical device or system 2509 comprises a mobile base unit and one or more EMF sensors.

In some embodiments, the medical device or system 2509 comprises a mobile base unit, one or more EMF sensors, and a shield for shielding ambient electromagnetic noise. In some embodiments, a mobile base unit includes wheels or a track upon which the mobile base unit is moved on a surface.

FIG. 10 depicts an example platform architecture that may be deployed through an environment, such as platform 2500 depicted in FIG. 9. The example platform architecture includes users 2610, portals 2620, PaaS services 2630, external services 2640, and API Gateway 2650. As depicted, users 2610 include global readers 2612, site users 2614, platform users 2616, and patients 2618. As depicted, portals 2620, includes GRP 2622, SRP 2624, operator portal 2626, internal portal 2627, billing portal 2628, and patient portal 2629. In some embodiments, PaaS services 2630 are deployed through as PaaS, such as Faraday. In some embodiments, the services 2630 are implemented as microservices. As depicted, PaaS services 2630 include user admin and authentication service 2632, global reader service 2633, site service 2634, EHR integration service 2635, signal processing service 2636, machine-learning service 2637, billing services 2638, and internal service 2639. In some embodiments, external services are services provided through third parties. As depicted, external services include SOS 2642, S3 2644, VPN 2646, and EMR 2648. In some embodiments, the API Gateway 2650 is an exposed set of API endpoints that coordinates a set of calls to different microservices.

In some embodiments, global readers 2612 include managed physicians with access to the GRP 2622. In some embodiments, site users 2614 include physicians, nurses, information technology (IT) personnel, administrators, and technicians with access to the SRP 2624 or the operator portal 2626. In some embodiments, platform users 2616 include IT personnel, customer service personnel, developers, administrators, and billing personnel with access to the internal portal 2627 or the billing portal 2628. In some embodiments, patients 2618 include patients with access to the patient portal 2629.

In some embodiments, the user admin and authentication service 2632 authenticates user credentials and provides access to other services in the API Gateway. In some embodiments, a user provides credentials (e.g., a username and password) to user admin and authentication service 2632 when logging into the described platform. In some embodiments, the user admin and authentication service 2632 returns a JSON Web Token (JWT) that allows the user to access other services. In some embodiments, the user admin and authentication service 2632 stores user information, such as name, email, phone number, National Provider Identifier (NPI), routing and account numbers, authorization level, and so forth. In some embodiments, a user is allowed access to various portals and services by the user admin and authentication service 2632 based on a respective user authorization level.

In some embodiments, the global reader service 2633 provides services to the global reader portals 2622. In some embodiments, global readers 2612 have access to their own GRP 2622. In some embodiments, cases from medical devices (e.g., CardioFlux) are routed to the appropriate specialty subset of readers within specified time slots, in the form of, for example, email or text, based on the reader's preference. The depicted architecture 2600 allows sites to take the burden off their on-site physicians and outsource readings without providing readers with access to Patient Health Information. In some embodiments, scans are uniquely identified by a respective scan identifier and provide relevant site information. In some embodiments, based on volume in the queue of scans that need to be read, notifications are stratified to send cases based on how likely readers are to complete and submit interpretations in under a specified threshold (e.g., one hour). Interpretations may include scan quality assessment, diagnosis, and any other additional comments. In some embodiments, readers are provided in-depth trainings and certifications prior to being registered onto the platform and being allowed to read.

In some embodiments, the site service 2634 provides patient information, scan interpretations and addendums received from global readers, access to customer service, an option to interface directly with global readers who have interpreted specific scans, and general support for SRP 2624. In some embodiments, through the site service 2634 user of sites can view all patient information that would otherwise be accessed directly from the EHR, with the addition of full dynamic reports for an integrated device, such as CardioFlux. In some embodiments, the site service 2634 allows site administrators to assign levels of visibility based on user assignments that can be made for each new profile. User assignments may include physicians (e.g., with a full view of all patient information), technicians (e.g., that can access the operator portal), and information technology (e.g., that can submit service tickets on a device). In some embodiments, a users' visibility can be assigned and edited within an administrator view. In some embodiments, pushes to credential editing can be obtained (e.g., forgot my password).

In some embodiments, the EHR integration service 2635 provides integration services for the employed PaaS. In some embodiments, the employed PaaS integrates with the integration service 2635 to extract information in relation to a patient's use of a medical device. This information includes, but is not limited to, a patient's demographic, insurance, diagnoses, conditions and medical history. In some embodiments, this information is used and displayed throughout the applicable portals. In some embodiments, the employed PaaS integrates with the integration service 2635 to push interpretations from Physicians back into the EHR. In some embodiments, information, such as interpretations, addendums, scan details and global reader identifying information is synthesized in a report. In some embodiments, such a report is generated directly within the EHR where physicians on-site with a device, such as CardioFlux, can access the information without adaptations or interruptions to their current workflow. In some embodiments, the employed PaaS integrates with the integration service 2635 to allow on-site physicians to also order scans, such as MRIs, CTs, stress tests and custom scans, in tandem with hospital techs being able to operate associated medical devices with prefilled patient data fields. Such integration allows for devices to seamlessly function within new sites, with minimal training and outside consultation. In some embodiments, the employed PaaS integrates with the integration service 2635 to populate information needed for filing insurance claims. At the end of the scan process, much of this information may been collected, but additional information, such as patient insurance information, provider and reader NPI information, reason for procedure, and other related procedures, can also be collected.

In some embodiments, the signal processing service 2636 processes recording data sent from the medical devices, such as CardioFlux. In some embodiments, signal processing service 2636 includes two pipelines—a processing pipeline and a signal previewing pipeline. In some embodiments, signal processing service 2636 includes two additional libraries—an Interpolation Library and Quantification Library. In some embodiments, a signal previewing script runs in the Signal Previewing Pipeline—this component generates a preview of the cardiac signal after a threshold amount of data is collected, (e.g., after 60 seconds of data collection or a set number of bytes). In some embodiments, this preview is shown in the operator portal 2626, which is discussed at length below. In some embodiments, a signal processing script runs in the signal processing pipeline. In some embodiments, this component generates the processed cardiac signal once a recording is complete and then quantifies the resulting magnetic field map. In some embodiments, the interpolation library, used by the Signal Processing Pipeline, handles interpolation of sensors in the final recording and is part of the signal quality determination process. In some embodiments, the parameter quantification library is used by the signal processing pipeline to handle the delineation of the T-wave and the quantification of the magnetic field map. In some embodiments, these components run on AWS Elastic Compute Cloud (EC2) instances and are deployed in Docker containers. In some embodiments, the Signal Processing Server is responsible for generating signal previews for the operator, generating the final processed signal, signal denoising, beat segmentation, cycle averaging, ensuring signal quality and magnetic field map generation, quantification and parameterization. In other device implementations, image/signal processing can be customized with a set of predefined protocols requested by device manufacturers.

In some embodiments, the machine-learning service 2637 includes an artificial neural network (ANN). In some embodiments, the ANN is provided a goal to determine how well it can reconstruct the repolarization magnetic field time series images. In some embodiments, the ANN is trained and generates high-quality reconstruction of normal repolarization (ST-T) segments. The hypothesis follows as such: the higher the reconstruction error, the more likely the patient's repolarization period is indicative of abnormal activity. In some embodiments, the ANN is trained using samples and validated to minimize the reconstruction error. In some embodiments, to test the efficacy of the ANN, cases are presented that the network has not seen. Based on this method, a scoring method can be devised. In some embodiments, the scoring method ranges from 0 to 5, when 3 or above represents acute cardiac abnormalities.

In some embodiments, the billing service 2638 automatically generates billing information. In some embodiments, EHR integration is integral to enable the billing functions of the PaaS, as most of the information that is needed to fill out insurance reimbursement forms can be found in hospital EHR systems. In some embodiments, this data is being collected throughout the workflow, and at completion of a scan, an internal billing analyst is presented with an auto-populated PDF form (e.g., CMS 1500 or UB-04) with patient demographic information, procedure codes and explanations, insurance information, and care provider information. In some embodiments, two forms are generated to receive reimbursement: one for the facility use of the device, and another for the physician read and interpretation of the scan data. In some embodiments, these claims are sent to the respective insurer (Center for Medicare & Medicaid Services, or other private insurer) and the claims process is tracked. In some embodiments, the internal billing analysts can add/modify information on this form, update the tracking process in the reimbursement lifecycle, and close any claims in the process. This service streamlines the billing process for the convenience of the care provider, institution, and the patient.

In some embodiments, the internal service 2639 enables IT administration functions and handles overall user and site administration. For example, the internal service 2639 may handle create, read, update, and delete (CRUD) functions for sites (hospitals), hospital admin users, and hospital usage statistics. In some embodiments, the internal service 2639 is also used to manage the registration and verification of global readers used for the telehealth aspects of the PaaS Analytical Cloud.

In some embodiments, each of the portals 2620 provides subsets of users' visibility to the data and/or requires access fields. In some embodiments, the GRP 2622 is deployed separately for each managed physicians. In some embodiments, the GRP 2622 provides notifications to physicians when scans are completed, a window to interpret these scans, and submission back to an original site. In some embodiments, through the GRP 2622, physicians are able to modify the times they want to be notified through their active hours settings. For example, physicians can completely turn off their notifications or change how they receive these alerts (e.g., text or email). In addition, changes to username, password, email, and phone number can be made within the global reader “Settings” tab. In some embodiments, the GRP 2622 provides a scan log for physicians that documents previous interpretations and addendums and allows for completion and submission of the documents. In some embodiments, each scan available in the GRP 2622 has a unique scan identifier as well as the ordering physician's name, site and phone number for easy access of readers. In some embodiments, global readers are able to access customer service within their respective portal.

In some embodiments, the SRP 2624 provides a list of patients that have taken a scan, such as a CardioFlux scan. In some embodiments, patient's information is auto-filled from information linking back to the EHR. In some embodiments, interpretations and addendums made from global readers can be viewed in the SRP 2624. In some embodiments, users accessing SRP 2624 can change their account settings, which allows them to alter their active hours and receive alerts based on the patients they created orders for. In some embodiments, physicians using their respective SRP 2624 can request addendums from global readers on any previous scan that has been submitted. In some embodiments, the administrator view of the site portal provides the assignment of specific users; provides further information of site details, such as number of users, number of scans, and so forth; and helps others with credential information, such as forgot password and/or username. In some embodiments, the SRP 2624 includes a customer service portal, where users can chat live with a representative, email from within the portal to track individual cases or directly call a support line. In some embodiments, a user can access the customer service portal and a self-service forum through the SRP 2624. In some embodiments, a self-service center provides different levels of support ranging from the platform to the device for technicians needing it. In some embodiments, access to a SRP 2624 and levels of visibility are assigned through a site administration portal. Based on the site administration's discretion, physicians, technicians, nurses, residents, and so forth can have access to the SRP 2624.

In some embodiments, the operator portal 2626 is accessed from a desktop that controls the physical device. In some embodiments, the operator portal 2626 is used to collect, analyze, and display the magnetic field image data. From this portal operators can: activate and control medical devices, such as CardioFlux (including bed insertion and data acquisition modules), create or select a pre-existing patient (EHR integration will fill out patient information once initial fields are filled), collect magnetic field image data and send confirmed data to the site portal for processing and future use. In some embodiments, accepting magnetic field images as being of adequate quality automatically notifies the GRP 2622 that there is a scan waiting to be read. In some embodiments, rejecting these images allows an operator to run the scan again or cancel the administration of the scan. In some embodiments, within the account settings, operators can also specify which alerts they wish to receive (e.g., physician orders scan, global reader rejects a scan due to quality, and so forth) and edit where they receive these alerts. In some embodiments, operators also have access to the customer service forum mentioned above. In some embodiments, operator visibility allows users to also access and create hardware tickets (for any issues with the physical device) that are directly posted.

In some embodiments, the internal portal 2627 has users ranging from administrators, IT, customer service, and developers. In some embodiments, much like in the SRP 2624, administrators can create accounts and assign users to different roles, which provide varying levels of access throughout the portal. In some embodiments, IT and customer service can view tickets that are filed and receive specific notifications to more closely monitor specific sites. Each ticket can be left unresolved, while it is being handled, or closed once there is a resolution from the user that filed the ticket. In some embodiments, tickets, customer complaints, calls and emails can also be tracked and viewed in Microsoft® Dynamics, as it is integrated with the customer service vendor's page. Developers can be flagged by customer service representatives based on the issue that needs to be solved. In some embodiments, the internal portal 2627 provides analytics on each user that has been created, which portals they have access to, and critical statistics depending on the user base (e.g., average time per scan for global readers, monthly scans for site portals, number of completed claims for billing portals, patient dialogue for patient portals, and so forth).

In some embodiments, internal billing analysts have access to a separate billing portal 2628. In some embodiments, the billing portal 2628 includes information on each claim that an individual has completed. In some embodiments, much like the scan log, the billing portal 2628 includes a claim log where relevant information regarding a patient and their provider are provided. In some embodiments, analysts can change the status of each claim as it is processed. Moreover, as with global readers, billing analysts can control which notifications they receive (based on each claim update) and how they receive them (phone/text). For example, based on each set of unique codes, analysts can choose exactly which follow-up information is required to most effectively file follow-ups to claims. In some embodiments, draft templates for relevant follow-ups can be found under “templates” in addition to best practices to submit each claim. This information can also be found in the customer service tab, with the self-service forum. This information, including general portal features and FAQs, can also be found here. In some embodiments, the billing portal 2628 displays billing analytics as they pertain to successful cases, pending cases, rejected cases, and so forth.

In some embodiments, when a patient has taken a scan from a monitored medical device, such as described above, they are given a unique set of credentials (e.g., based on a scan identifier) to view all follow-ups in reference to their claim. In some embodiments, the patient portal 2629 provides these patients updates in the status of the claim that are, for example, filed on the hospital's behalf. In some embodiments, in account settings, patients can view and select alerts (e.g., submissions, re-submissions, acceptances, and so forth). In some embodiments, through the patient portal 2629, patients can choose to interact directly with a customer support forum, which may include self-service search, live chat with representatives, email and call.

### EMF Sensing Devices and Systems

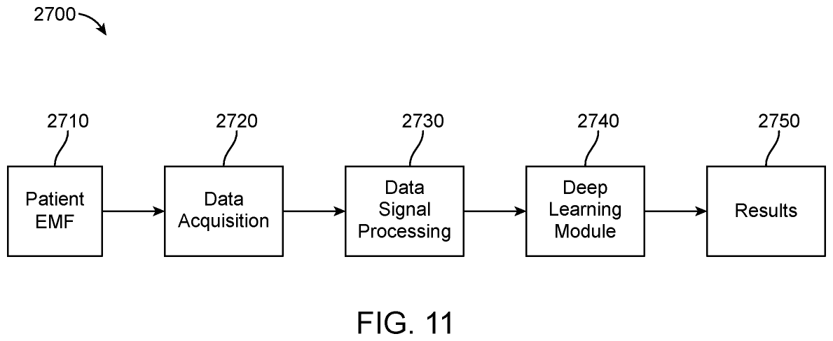

FIG. 11 depicts a schematic representation of an exemplary medical device or system 300 for sensing and/or analyzing an EMF. In some embodiments, medical device or system 300 can be deployed in an environment, such as platform 2500, and include medical device or system 2509 of FIG. 9. It should be also understood that any medical device or system is suitable for use with the platforms described herein including and not limited to medical imaging and medical monitoring systems. Generally, any medical device or system that receives, generates, or senses medical data from an individual is suitable for use in addition to or in place of the medical device or system 2700 in various embodiments of the platforms described herein.

As shown in FIG. 11, an EMF 2710, which is associated with an individual (e.g., an EMF generated by a current traveling through myocardium), is acquired from the EMF sensor or sensors 2720 (e.g., a sensor array). The data is then processed, optionally filtered and analyzed by a signal processing module 2730. A signal processing module 2730 in some embodiments removes noise if any from the sensed EMF signal and extracts information from the data. The processed data is then fed into the deep learning module 2740 that, in some embodiments, includes dilated convolutional neural networks. The deep learning module detects, for example, ischemia and localizes to a particular region in an organ and provides these as results 2750.

An operation of a device or system may be controlled using a software User Interface (UI). In some cases, a software UI may be installed on site, on a provided accessory computer. The use of the device may be prescribed by a medical professional such as a physician to determine more information regarding a subject's condition. Within the software user interface, User preferences and acquisition parameters may be chosen, including a sampling rate and an axis operation of the device or system. From the software user interface, magnetic field signals from a subject, such as signals corresponding to a subject's heart, can be displayed and can be saved to a file. The device or system may be used to measure cardiac electrical activity, creating waveforms similar to electrocardiograph recordings which may demonstrate points of interest in a cardiac cycle.

A device or system may be constructed to overcome tradeoffs associated with older SQUID devices to maximize clinical utility, while remaining cost-effective and technician-friendly. A device or system may present no physical risk to a subject and may be an adjunctive tool employed in addition to a second medical procedure or clinical measurement in order to aid a physician to provide more detailed information regarding a subject's condition. These inventions are the first of their kind using optically pumped magnetometers for measurements of biomagnetic measurements. A device or system as described herein is the first example of OPMs used in a compact shield based design. A device or system as described herein may be the first entirely self-contained biomagnetic detection system that utilizes this compact shield design. A device or system as described herein is the first example of a mobile cart and bedside deployable unit for biomagnetic measurements.

Traditional OPMs that have a desired level of sensitivity for biomagnetic measurements are understood to have a dynamic range which necessarily limits their use to low magnetic field environments, wherein ambient noise is generally less than about 100 nanotesla. The earth's magnetic field is naturally present everywhere on earth, and the amplitude is about 50 microtesla (about 500 times greater than the ceiling of operation of a device as described herein).

To combat ambient noise, some embodiments of the devices and systems described herein provide an electromagnetic shield comprising a metal alloy (e.g., permalloy or mumetal), which when annealed in a hydrogen furnace typically have exceptionally high magnetic permeability. When formed into a shielding barrier or chamber, the permeable alloy absorbs magnetic field signals and provides a pathway for the magnetic signals to travel along (i.e., on the surface of or within the body of the alloy) so as to shield the embodiments of the devices and systems that include these shields.

In some embodiments, a device or system as described herein comprises a shield in the form of a room or chamber configured to minimize interior magnetic fields within the chamber. In some embodiments, the room or chamber may have one or more openings. In some embodiments, the opening may be a door.

A patient may enter the shielded room or chamber through a door. In some embodiments, a patient walks into the shielded room or chamber. In some embodiments, a patient stands in the shielded room or chamber during device use. In some embodiments, a patient enters the shielded room or chamber with the aid of a mobility device. In some embodiments, a mobility device is a manual wheelchair, power wheelchair, power scooter, hospital bed, crib, bassinet, stretcher, walker, cane, braces, or crutches. In some embodiments, the patient remains sitting or lying in the mobility device during device use. For example, a patient may be in a wheelchair. In some embodiments, the patient is wheeled into the shielded room or chamber through a door. In some embodiments, the patient remains seated in a wheelchair during device use. In another example, a patient may be wheeled into the shielded room or chamber on a hospital bed. The patient may remain lying in the hospital bed during device use. In some embodiments, a patient is positioned or loaded outside of the shielded room or chamber. In some embodiments, a patient is positioned or loaded inside of the shielded room or chamber. In some embodiments, a mobility device, like a wheelchair or hospital bed, may be adjusted or repositioned prior to device use. In some embodiments, a mobility device, like a wheelchair or hospital bed, may be adjusted or repositioned during device use. In some embodiments, a patient be repositioned within the shielded room or chamber for application of a different module. For example, a patient may initially be positioned in a seated position for a cardiac module and then reposition into a standing position for a neurological module.

During device use, a flexible jointed arm with x-y-z translational movement (may be able to occupy any point within a semicircle defined by total arm length at extension) may be used to position an array of n-optically pumped magnetometers in a wide range of geometries on or proximally above a portion of a subject (such as a subject's chest, head, or other organ) using a set standard operating procedure based on an organ of interest, a condition or disease of interest, or a combination thereof. After this point, the sensor array may be turned on and at least a portion of the subject, at least a portion a mobility device, or a combination thereof may enter the shielded room or chamber. Using a provided computer application, fast calibration of the sensors may occur, and then the magnetic field of the organ of interest can be displayed, can be recorded, or a combination thereof for immediate or later analysis. Electronic drivers for the sensors may be housed either underneath the shield portion of the device, or may be housed in an adjacent cart with computer control. The system may also involve a touch screen computer interface (such as a graphical user interface) housed on a side of the device itself, or on said adjacent cart.

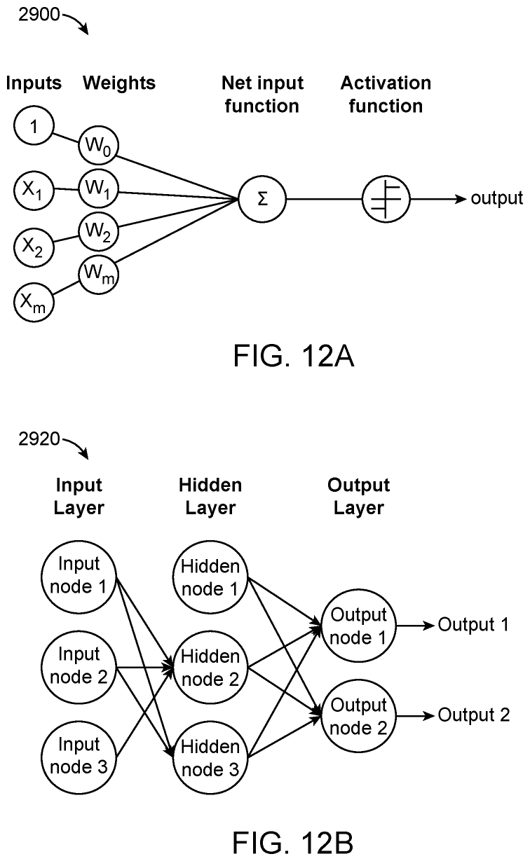

In some embodiments, an ANN, such as the ANN depicted in FIG. 12A, may be employed within the machine-learning service 2637 of FIG. 10 comprised of a series of layers termed “neurons.” FIG. 12A depicts typical neuron 2900 in an ANN. As illustrated in FIG. 12B, in embodiments of ANNs 2920, there is an input layer to which data is presented; one or more internal, or “hidden,” layers; and an output layer. A neuron may be connected to neurons in other layers via connections that have weights, which are parameters that control the strength of the connection. The number of neurons in each layer may be related to the complexity of the problem to be solved. The minimum number of neurons required in a layer may be determined by the problem complexity, and the maximum number may be limited by the ability of the neural network to generalize. The input neurons may receive data from data being presented and transmit that data to the first hidden layer through connections' weights, which are modified during training. The first hidden layer may process the data and transmit its result to the next layer through a second set of weighted connections. Each subsequent layer may “pool” the results from the previous layers into more complex relationships. In addition, whereas conventional software programs require writing specific instructions to perform a function, neural networks are programmed by training them with a known sample set and allowing them to modify themselves during (and after) training so as to provide a desired output such as an output value. After training, when a neural network is presented with new input data, it is configured to generalize what was “learned” during training and apply what was learned from training to the new previously unseen input data in order to generate an output associated with that input.

In some embodiments of a machine learning software module as described herein, a machine learning software module comprises a neural network such as a deep convolutional neural network. In some embodiments in which a convolutional neural network is used, the network is constructed with any number of convolutional layers, dilated layers or fully connected layers. In some embodiments, the number of convolutional layers is between 1-10 and the dilated layers between 0-10. In some embodiments, the number of convolutional layers is between 1-10 and the fully connected layers between 0-10.

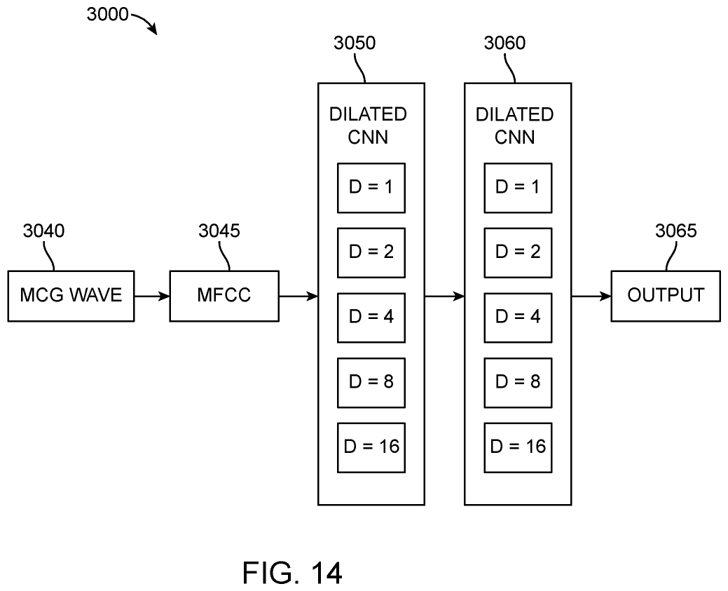

FIG. 14 depicts a flow chart 3000 representing the architecture of an exemplary embodiment of a machine learning software module, which may be employed within the machine-learning service 2637 of FIG. 10. In this exemplary embodiment, raw EMF 3040 of the individual is used to extract the MFCC features 3045 which are fed into the deep learning module. The machine learning software module comprises two blocks of Dilated Convolutional neural networks 3050, 3060. Each block has 5 dilated convolution layers with dilation rates D=1, 2, 4, 8, 16. The number of blocks and the number of layers in each block can increase or decrease, so it is not limited to the configuration portrayed in FIG. 14.

### Machine Learning

a. Training Phase

A machine learning software module as described herein is configured to undergo at least one training phase wherein the machine learning software module is trained to carry out one or more tasks including data extraction, data analysis, and generation of output 665.

In some embodiments of the software application described herein, the software application comprises a training module that trains the machine learning software module. The training module is configured to provide training data to the machine learning software module, said training data comprising, for example, EMF measurements and the corresponding abnormality data. In additional embodiments, said training data is comprised of simulated EMF data with corresponding simulated abnormality data. In some embodiments of a machine learning software module described herein, a machine learning software module utilizes automatic statistical analysis of data in order to determine which features to extract and/or analyze from an EMF measurement. In some of these embodiments, the machine learning software module determines which features to extract and/or analyze from an EMF based on the training that the machine learning software module receives.

In some embodiments, a machine learning software module is trained using a data set and a target in a manner that might be described as supervised learning. In these embodiments, the data set is conventionally divided into a training set, a test set, and, in some cases, a validation set. A target is specified that contains the correct classification of each input value in the data set. For example, a set of EMF data from one or more individuals is repeatedly presented to the machine learning software module, and for each sample presented during training, the output generated by the machine learning software module is compared with the desired target. The difference between the target and the set of input samples is calculated, and the machine learning software module is modified to cause the output to more closely approximate the desired target value. In some embodiments, a back-propagation algorithm is utilized to cause the output to more closely approximate the desired target value. After a large number of training iterations, the machine learning software module output will closely match the desired target for each sample in the input training set. Subsequently, when new input data, not used during training, is presented to the machine learning software module, it may generate an output classification value indicating which of the categories the new sample is most likely to fall into. The machine learning software module is said to be able to “generalize” from its training to new, previously unseen input samples. This feature of a machine learning software module allows it to be used to classify almost any input data which has a mathematically formulatable relationship to the category to which it should be assigned.

In some embodiments of the machine learning software module described herein, the machine learning software module utilizes an individual learning model. An individual learning model is based on the machine learning software module having trained on data from a single individual and thus, a machine learning software module that utilizes an individual learning model is configured to be used on a single individual on whose data it trained.

In some embodiments of the machine training software module described herein, the machine training software module utilizes a global training model. A global training model is based on the machine training software module having trained on data from multiple individuals and thus, a machine training software module that utilizes a global training model is configured to be used on multiple patients/individuals.

In some embodiments of the machine training software module described herein, the machine training software module utilizes a simulated training model. A simulated training model is based on the machine training software module having trained on data from simulated EMF measurements. A machine training software module that utilizes a simulated training model is configured to be used on multiple patients/individuals.

In some embodiments, the use of training models changes as the availability of EMF data changes. For instance, a simulated training model may be used if there are insufficient quantities of appropriate patient data available for training the machine training software module to a desired accuracy. This may be particularly true in the early days of implementation, as few appropriate EMF measurements with associated abnormalities may be available initially. As additional data becomes available, the training model can change to a global or individual model. In some embodiments, a mixture of training models may be used to train the machine training software module. For example, a simulated and global training model may be used, utilizing a mixture of multiple patients' data and simulated data to meet training data requirements.

Unsupervised learning is used, in some embodiments, to train a machine training software module to use input data such as, for example, EMF data and output, for example, a diagnosis or abnormality. Unsupervised learning, in some embodiments, includes feature extraction which is performed by the machine learning software module on the input data. Extracted features may be used for visualization, for classification, for subsequent supervised training, and more generally for representing the input for subsequent storage or analysis. In some cases, each training case may consist of a plurality of EMF data.

Machine learning software modules that are commonly used for unsupervised training include k-means clustering, mixtures of multinomial distributions, affinity propagation, discrete factor analysis, hidden Markov models, Boltzmann machines, restricted Boltzmann machines, autoencoders, convolutional autoencoders, recurrent neural network autoencoders, and long short-term memory autoencoders. While there are many unsupervised learning models, they all have in common that, for training, they require a training set consisting of biological sequences, without associated labels.

A machine learning software module may include a training phase and a prediction phase. The training phase is typically provided with data in order to train the machine learning algorithm. Non-limiting examples of types of data inputted into a machine learning software module for the purposes of training include medical image data, clinical data (e.g., from a health record), encoded data, encoded features, or metrics derived from an electromagnetic field. Data that is inputted into the machine learning software module is used, in some embodiments, to construct a hypothesis function to determine the presence of an abnormality. In some embodiments, a machine learning software module is configured to determine if the outcome of the hypothesis function was achieved and based on that analysis make a determination with respect to the data upon which the hypothesis function was constructed. That is, the outcome tends to either reinforce the hypothesis function with respect to the data upon which the hypothesis functions was constructed or contradict the hypothesis function with respect to the data upon which the hypothesis function was constructed. In these embodiments, depending on how close the outcome tends to be to an outcome determined by the hypothesis function, the machine learning algorithm will either adopt, adjust, or abandon the hypothesis function with respect to the data upon which the hypothesis function was constructed. As such, the machine learning algorithm described herein dynamically learns through the training phase what characteristics of an input (e.g., data) are most predictive in determining whether the features of a patient EMF display any abnormality.

For example, a machine learning software module is provided with data on which to train so that it, for example, is able to determine the most salient features of a received EMF data to operate on. The machine learning software modules described herein train as to how to analyze the EMF data, rather than analyzing the EMF data using pre-defined instructions. As such, the machine learning software modules described herein dynamically learn through training what characteristics of an input signal are most predictive in determining whether the features of an EMF display any abnormality.

In some embodiments, the machine learning software module is trained by repeatedly presenting the machine learning software module with EMF data along with, for example, abnormality data. The term “abnormality data” is meant to comprise data concerning the existence or non-existence of an abnormality in an organ, tissue, body, or portion thereof. Any disease, disorder or condition associated with the abnormality is included in the abnormality data if available. For example, information concerning a subject displaying symptoms of hypertension, ischemia or shortness of breath is included as abnormality data. Information concerning a subject's lack of any irregular health condition is also included as abnormality data. In the case where EMF data is generated by computer simulation, the abnormality data may be used as additional data being used to simulate the organ, tissue, body, or portion thereof. In some embodiments, more than one abnormality is included in the abnormality data. In additional embodiments, more than one condition, disease or disorder is included in the abnormality data.

In some embodiments, training begins when the machine learning software module is given EMF data and asked to determine the presence of an abnormality. The predicted abnormality is then compared to the true abnormality data that corresponds to the EMF data. An optimization technique such as gradient descent and backpropagation is used to update the weights in each layer of the machine learning software module so as to produce closer agreement between the abnormality probability predicted by the machine learning software module, and the presence of the abnormality. This process is repeated with new EMF data and abnormality data until the accuracy of the network has reached the desired level. In some embodiments, the abnormality data additionally comprises the type and location of the abnormality. For example, the abnormality data may indicate that an abnormality is present, and that said abnormality is an ischemia of the left ventricle of the heart. In this case, training begins when the machine learning software module is given the corresponding EMF data and asked to determine the type and location of the abnormality. An optimization technique is used to update the weights in each layer of the machine learning software module so as to produce closer agreement between the abnormality data predicted by the machine learning software module, and the true abnormality data. This process is repeated with new EMF data and abnormality data until the accuracy of the network has reached the desired level. In some embodiments, the abnormality data additionally comprises a known resulting or related disease, disorder or condition associated with an identified abnormality. For example, the abnormality data may indicate that the subject possesses an atrial flutter and arterial coronary disease. In cases such as this, training begins when the machine learning software module is given the corresponding EMF data and asked to determine the presence of a condition, disorder or disease. The output data is then compared to the true abnormality data that corresponds to the EMF data. An optimization technique is used to update the weights in each layer of the machine learning software module so as to produce closer agreement between the abnormality probability predicted by the machine learning software module, and the actual abnormality. This process is repeated with new EMF data and abnormality data until the accuracy of the network has reached the desired level. Following training with the appropriate abnormality data given above, the machine learning module is able to analyze an EMF measurement and determine the presence of an abnormality, the type and location of said abnormality and the conditions associated with such.

In some embodiments of the machine learning software modules described herein, the machine learning software module receives EMF data and directly determines the abnormality probability of the subject, wherein the abnormality probability comprises the probability that the EMF measurement is associated with the abnormality of the subject.

In some embodiments, the machine learning software module is trained on a single continuous EMF measurement with corresponding abnormality data over a period of time. This can greatly increase the amount of training data available to train a machine learning software module. For example, in an EMF recording consisting of N continuous 10-second segments with accompanying abnormality data, one can generate at least N*N pairs of such segments to train on.

In some embodiments, an individual's abnormality data is inputted by the individual of the system. In some embodiments, an individual's abnormality data is inputted by an entity other than the individual. In some embodiments, the entity can be a healthcare provider, healthcare professional, family member or acquaintance. In additional embodiments, the entity can be the instantly described system, device or an additional system that analyzes EMF measurements and provides data pertaining to physiological abnormalities.

In some embodiments, a strategy for the collection of training data is provided to ensure that the EMF measurements represent a wide range of conditions so as to provide a broad training data set for the machine learning software module. For example, a prescribed number of measurements during a set period of time may be required as a section of a training data set. Additionally these measurements can be prescribed as having a set amount of time between measurements. In some embodiments, EMF measurements taken with variations in a subject's physical state may be included in the training data set. Examples of physical states include accelerated heart rate and enhanced brain signaling. Additional examples include the analysis of a subject's EMF data under the influence of medication or during the course of medical treatment.

In some embodiments, training data may be generated by extracting random overlapping segments of EMF measurements performed by the subject. In some embodiments, training examples can be provided by measurement recordings, models or algorithms that are independent of the subject. Any mixture or ratio of subject and non-subject training measurements can be used to train the system. For example, a network may be trained using 5 EMF segments extracted from a subject's measurements, and 15,000 EMF segments taken from another subject's recordings. Training data can be acquired using two different methods. The first method is to directly measure the EMF measurements over a subject's chest. The second method involves creating an accurate electro-anatomical model of the heart. This electro-anatomical model can be used to generate EMF measurements of both healthy and diseased subjects. The measurements are acquired by applying the Biot-Savart Law. This calculates the magnetic field vector at a given point in space, caused by a specific movement of current. After the EMF measurements have been acquired or calculated, they are fed into the network with a classification label, describing both the presence and location of diseased tissue.

In general, a machine learning algorithm is trained using a large patient database of medical image and/or clinical data and/or encoded data from one or more EMF measurements and/or any features or metrics computed from the above said data with the corresponding ground-truth values. The training phase constructs a transformation function for predicting probability of an abnormality in an unknown patient's organ, tissue, body, or portion thereof by using the medical image and/or clinical data and/or encoded data from the one or more EMF measurements and/or any features or metrics computed from the above said data of the unknown patient. The machine learning algorithm dynamically learns through training what characteristics of an input signal are most predictive in determining whether the features of a patient EMF data display any abnormality. A prediction phase uses the constructed and optimized transformation function from the training phase to predict the probability of an abnormality in an unknown patient's organ, tissue, body, or portion thereof by using the medical image and/or clinical data and/or encoded data from the one or more EMF measurements and/or any features or metrics computed from the above said data of the unknown patient.

b. Prediction Phase

Following training, the machine learning algorithm is used to determine, for example, the presence or absence of an abnormality on which the system was trained using the prediction phase. With appropriate training data, the system can identify the location and type of an abnormality, and present conditions associated with such abnormality. For example, an EMF measurement is taken of a subject's brain and appropriate data derived from the EMF measurement is submitted for analysis to a system using the described trained machine learning algorithm. In these embodiments, a machine learning software algorithm detects an abnormality associated with epilepsy. In some embodiments, the machine learning algorithm further localizes an anatomical region associated with an abnormality such as, for example, localizing an area of the brain of an individual associated with epilepsy in the individual based on an EMF measurement of an individual.

An additional example, a subject is known to possess arterial ischemia and has EMF measurements recorded before and after treatment with a medication. The medical image and/or clinical data and/or encoded data from the EMF measurements and/or features and/or metrics derived from the said data are submitted for analysis to a system using the described trained machine learning algorithm in order to determine the effectiveness of the medication on abnormal blood flow using the prediction phase.

The prediction phase uses the constructed and optimized hypothesis function from the training phase to predict the probability of an abnormality in an unknown patient's organ, tissue, body, or portion thereof by using the medical image and/or clinical data and/or encoded data from the EMF measurements and/or any features or metrics computed from the above said data of the unknown individual.

In some embodiments, in the prediction phase, the machine learning software module can be used to analyze data derived from its EMF measurement independent of any system or device described herein. In these instances, the new data recording may provide a longer signal window than that required for determining the presence of a subject's abnormality. In some embodiments, the longer signal can be cut to an appropriate size, for example 10 seconds, and then can be used in the prediction phase to predict the probability of an abnormality of the new patient data.

In some embodiments, a probability threshold can be used in conjunction with a final probability to determine whether or not a given recording matches the trained abnormality. In some embodiments, the probability threshold is used to tune the sensitivity of the trained network. For example, the probability threshold can be 1%, 2%, 5%, 10%, 15%, 20%, 25%, 30%, 35%, 40%, 45%, 50%, 55%, 60%, 65%, 70%, 75%, 80%, 85%, 90%, 95%, 98% or 99%. In some embodiments, the probability threshold is adjusted if the accuracy, sensitivity or specificity falls below a predefined adjustment threshold. In some embodiments, the adjustment threshold is used to determine the parameters of the training period. For example, if the accuracy of the probability threshold falls below the adjustment threshold, the system can extend the training period and/or require additional measurements and/or abnormality data. In some embodiments, additional measurements and/or abnormality data can be included into the training data. In some embodiments, additional measurements and/or abnormality data can be used to refine the training data set.

### Input Data

As described herein, a machine learning software module is typically provided with data (input) in order to train the machine learning software module as to how to analyze an EMF to determine, for example, the presence of an abnormality. Input data is also used by a machine learning software module to generate an output.

An input to a machine learning algorithm as described herein, in some embodiments, is data transmitted to the machine learning algorithm by a device or a system which includes an EMF sensor. In some embodiments of the devices, systems, software, and methods described herein, data that is received by a machine learning algorithm software module from an electromagnetic sensor as an input may comprise EMF data expressed in a standard unit of measurement such as, for example, Tesla.

In some embodiments, sensed EMF data comprises an overall or total EMF generated by a body of an individual based on numerous different currents generated by the body of the individual. That is, in some embodiments, one or more EMF sensors sense an EMF that comprises an EMF associated with an entire individual and is not specific to a single organ, tissue, body, or portion thereof. Likewise, in some embodiments, an EMF is sensed from an individual that is associated with a portion of the individual, but not specific to a single organ, tissue, body, or portion thereof.

In some embodiments, sensed EMF data comprises an EMF that is in proximity to an individual or a portion of the body of the individual and comprises an EMF associated with a single organ, organ system, or tissue. For example, in some embodiments, one or more EMF sensors are positioned in proximity to a chest of an individual and sense an EMF associated with a heart of the individual. For example, in some embodiments, one or more EMF sensors are positioned in proximity to a head of an individual and sense an EMF associated with a brain of the individual. For example, in some embodiments, one or more EMF sensors are positioned in proximity to a chest of an individual and sense an EMF associated with a cardio-pulmonary system (i.e., the heart and lungs).

In some embodiments, a machine learning software module is configured to receive an encoded length of EMF data as an input and to determine the window length of the input data. For example, an input to a machine learning software module in some embodiments described herein is 100 seconds of encoded EMF data, and the machine learning software module selects a 10 second segment within the 100 second data sample for examination. In some embodiments, the input is segmented into multiple inputs, any number of which is analyzed independently. Any number of these analyses may be used to determine the final output.

In some embodiments, a device, system, or method as described herein is configured to sense and/or receive data comprising data associated with an individual. Data is sensed, in some embodiments, by an electromagnetic field sensor that is a component of a device, system, or method described herein. Data is received, in some embodiments, by transmission of data to a software algorithm as described herein by a source other than an EMF that is a component of a device, system, or method that also includes the software algorithm. That is, data, in some embodiments, is received from a source remote from the device, system, or method that includes the software algorithm. In some embodiments, data that is received comprises stored data. In some embodiments, data that is received comprises data that is generated by a software module. In general, sensed and/or received data comprises an input to a machine learning algorithm as described herein. An input is used to train a machine learning algorithm and/or is used by the machine learning algorithm to carry out an analysis or prediction.

Data as described herein comprises EMF data as well as other information associated with an individual. Non-limiting examples of data used as an input for a machine learning algorithm as described herein include a medical record (e.g., an electronic health record), a diagnosis, a lab value, a vital sign, a prognosis, an electrocardiogram, a radiology image (including ultrasound, CT scan, MRI, and X-ray), an electroencephalogram, and a pathology report. In some embodiments, two or more different types of data are combined and/or correlated by the software algorithms described herein.

EMF data, in some embodiments, is used to generate other types of data that are used by the software algorithms described herein. For example, EMF data, in some embodiments, is used to generate medical image data which, in some embodiments, is achieved using Magnetic Field Maps (MFM). In some embodiments, EMF data is used to generate medical image data using Pseudo-Current Density (PCD) maps. In some embodiments, EMF data is used to generate medical data using Spatio-Temporal Activation Graphs (STAG).

EMF data, in some embodiments, is used to generate clinical data such as MCG, MEG and MGG measurements.

In some embodiments, input to a software algorithm as described herein comprises EMF data which is encoded into some other form of data and the features or metrics computed from the encoded data such as, for example, MFCC.

In some embodiments, input to a software algorithm as described herein is generated by a computer. For example, in some embodiments, an input to a software algorithm as described herein comprises data generated by computer simulation. In some embodiments, a computer simulation generates an image or other representation of an organ or other tissue (including skin, bone, and blood). In some embodiments, a computer simulation generates an image or representation of a flow of a fluid such as, for example, blood, lymph, or bile. In some embodiments, a computer simulation generates an image or representation of a flow of an electric current. Non-limiting examples of additional inputs generated by a computer simulation include a medical record (e.g., an electronic health record), a diagnosis, a lab value, a vital sign, a prognosis, an electrocardiogram, a radiology image (including ultrasound, CT scan, MRI, and X-ray), an electroencephalogram, and a pathology report.

### Data Filtering

In some embodiments of the devices, systems, software, and methods described herein, data that is received by a machine learning algorithm software module from an electromagnetic sensor as an input may comprise EMF data that has been filtered and or modified. In some embodiments, filtering comprises a removal of noise or artifacts from a sensed electromagnetic field data. Artifacts or noise may comprise, for example, ambient electromagnetic signals that are sensed together with electromagnetic data sensed from an individual.

In some embodiments of the devices, systems, software, and methods described herein, sensed EMF data is filtered prior to and/or after transmission of said data to a processor. Filtering of sensed EMF data may, for example, comprise the removal of ambient signal noise from a sensed EMF data. Signal noise may, for example, comprise ambient EMF data generated by, for example, electronic devices, the earth's magnetosphere, electrical grids, or other individuals (i.e., not individuals whose EMF data is being targeted).

In some embodiments, sensed EMF data is converted to another form of data or signal which then undergoes a signal filtering process. In some embodiments, a device or system includes a processor including software that is configured to convert sensed EMF data to another form of data or signal. The process of converting sensed EMF data to another form of data or signal typically comprises an encoding process, wherein a first form of data is converted into a second form of data or signal.

In some embodiments, sensed EMF data is encoded into an audio signal which undergoes a filtering process. In some embodiments, sensed EMF data is encoded into an audio signal or alternatively, a signal having the morphology of an audio signal.

In some embodiments, sensed EMF data is encoded into an audio signal which is further processed into a Mel-Frequency Cepstrum from which one or more Mel-Frequency Cepstrum Coefficients (“MFCC”) are derived. Mel-Frequency Cepstrum (“MFC”) represents a short term power spectrum of a sound. It is based on a linear cosine transform of a log power spectrum on a nonlinear mel scale of frequency. Mel-frequency cepstral coefficients (“MFCCs”) collectively make up an MFC. These are derived from a type of cepstral representation of the audio. In MFC, frequency bands are equally spaced on the mel-scale as compared to the linearly-spaced frequency bands used in the normal cepstrum. These equally spaced frequency bands allows for better representation of audio.

In some embodiments, a sensed EMF signal is filtered by converting the sensed EMF data into an audio signal or a signal having the morphology of an audio signal wave, and then generating MFCCs.

MFCCs help in identifying the components of the audio signal that are able to differentiate between important content and background noise.

In general, steps for filtering an audio signal derived from sensed EMF data comprise: In a first step, the audio signal is framed into short frames. In a second step, the periodogram estimate of the power spectrum for each frame is calculated. In a third step, a mel filterbank is applied to the power spectrum and sums the energy in each filter. In a fourth step, the logarithm of all the filterbank energies is determined and the DCT of the log filterbank energies is calculated. In a fifth step, only the first 20 DCT coefficients are kept, and the rest are discarded.

Once filtered, the filtered data is transmitted to a machine learning algorithm for analysis. The algorithm described herein is capable of classifying and characterizing the physiological health of human body tissues. The algorithm is designed to analyze input data and determine the presence and location of diseased tissue in the organ(s) recorded by aforementioned sensors.

### Devices and Systems

In some embodiments EMF data is sensed using a device or system. In some embodiments, a device or system comprises one or more EMF sensors. In some of these embodiments, the device or system is configured to include a machine learning software module as described herein. In some of these embodiments, the device or system is configured to transmit a sensed EMF to a machine learning software module not included as part of the device or system. EMF data that is sensed using an electromagnetic sensor comprises electromagnetic data associated with a passage of a current through a cell, tissue, and/or organ of an individual, such as, for example, the heart of the individual. Generally, described herein are devices and systems that comprise digital processing devices.

In some embodiments of devices and systems described herein, a device and/or a system comprises a digital processing device configured to run a software application as described herein. In further embodiments, a digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected to a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, handheld computers, and tablet computers.

In some embodiments, the digital processing device includes an operating system configured to perform executable instructions. The operating system is, for example, software, including programs and data, which manages the device's hardware and provides services for execution of applications. Non-limiting examples of suitable operating systems include FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing.

In some embodiments, a digital processing device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes, optical disk drives, and cloud computing based storage. In further embodiments, the storage and/or memory device is a combination of devices such as those disclosed herein.

In some embodiments, the digital processing device includes a display to send visual information to a subject. In some embodiments, the digital processing device includes an input device to receive information from a subject. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In still further embodiments, the input device is a combination of devices such as those disclosed herein.

### EXAMPLES

Non-limiting examples of embodiments and elements of embodiments of the devices and systems described herein are as follows:

### Example 1—Operating Instructions

A magnetically shielded environment: comprises minimum outer dimensions of about 6 foot width x about 6 foot depth×about 7 foot height and minimum inner dimensions of about 5 foot width×about 5 foot depth×about 6 foot height. A magnetically shielded environment, in some embodiments, comprises a DC shielding factor of at least about 500 with minimum shielding factor of about 56 decibel (dB) from a bandwidth of from about 0.1 Hz to about 500 Hz at all points at least about 1 foot from each surface of a magnetically shielded environment.
A patient may enter the shielded room or chamber through a door. In some embodiments, a patient walks into the shielded room or chamber. In some embodiments, a patient stands in the shielded room or chamber during device use. In some embodiments, a patient enters the shielded room or chamber with the aid of a mobility device. In some embodiments, a mobility device is a manual wheelchair, power wheelchair, power scooter, hospital bed, crib, bassinet, stretcher, walker, cane, braces, or crutches. In some embodiments, the patient remains sitting or lying in the mobility device during device use. For example, a patient may be in a wheelchair. In some embodiments, the patient is wheeled into the shielded room or chamber through a door. In some embodiments, the patient remains seated in a wheelchair during device use. In another example, a patient may be wheeled into the shielded room or chamber on a hospital bed. The patient may remain lying in the hospital bed during device use. In some embodiments, a patient is positioned or loaded outside of the shielded room or chamber.

Setup: To setup a device for use, one or more of the following exemplary steps are carried out:
Ensure that device frame and sensor housing are located inside a magnetically shielded room or chamber.
Ensure that the control units are connected to a sensor housing and a device frame through one or more holes or throughputs of the magnetically shielded room or chamber.
Power on the computer interface and launch the software application (such as Maxwell).
Power on an Electronic Control Module.

Initiation: After a frame is in position, one or more sensors are activated to prepare for recording a signal, such as cardiac magnetic activity. To begin initiation, a user logs in to a software application (such as Maxwell) and selects the data acquisition module. If there is trouble with any of the steps below, the application is closed and attempts to reopen. If a problem does not go away, the computer interface is rebooted. To initiate a device for use, one or more of the following is adhered to:
Ensure connection to all sensors (such as 8 sensors) exists by checking sensor status in the data acquisition software user interface.
Initiate the autostart procedure through the software application by pressing “autostart” in the data acquisition software user interfaces. This process calibrates one or more sensors for use. Before continuing, ensure that the readiness indicator found in the software UI has turned green and the status reads “ready”.

Recording: After initiation is complete, the device is ready to capture a signal, such as a cardiac magnetic field data. To begin, one or more of the following is carried out:
Select the “acquire” button in the software application. Selecting this option plots the magnetic field collected from the sensors in a viewing window found on the acquisition software UI.
Ensure a collected magnetic field is characteristic of a signal, such as a cardiac electrical activity.
To save data to a file, select the “record” option. Select preferences for period length of data acquisition, file name, and file save location. Select “save” to begin saving to file. Application, in some embodiments, automatically cease saving after a selected amount of time that has elapsed. Files are named in accordance with institutional policy to protect subject identifying information.

Option for Additional Testing: After the use of a certain module within the device is complete, the patient may undergo another module. The patient may be repositioned or adjusted prior to the second module. Multiple modules may be used on the same patient in the same shielded room or chamber.

Power-down and Storage: After device use is complete, the system is powered down by following one or more of the following:
Close the application on the computer.
Power off the electronic control modules by turning the toggle switch to the “off” position.
Power off the computer.

After device use, the door of the magnetically shielded chamber or room may be opened. The patient may walk out of the shielded chamber or room. In some embodiments, the patient is wheeled out of the shielded chamber or room on a wheelchair or hospital bed.

### Example 2—Module for Magnetorelaxometry

The present disclosure provides systems and methods for conditioning a magnetic shield (e.g., locally or globally) using stimulation (e.g., magnetic, electrical, and/or mechanical), in order to create or maintain a stable magnetic field suitable for magneto-relaxometry (MRX) measurements. In some embodiments, a movable coil and magnetometer setup is used for site specific measurements of magnetorelaxometry measurements. In some embodiments, the site-specific measurements are operator positioned. In some embodiments, the movable coil excites tissue while sensors pick up relaxation curves, as applicable. This may include any combination of single, multiple, or continuous stimulation signals. The systems and methods may also comprise performing MRX measurements using the conditioned magnetic shield. In some embodiments, the magnetic shield comprises a mu metal shield.

Using systems and methods of the present disclosure, a biomagnetic detection platform is constructed for the detection of superparamagnetic nanoparticles via magneto-relaxometry (MRX) analysis. During this detection process, a 60-gauss magnetic field is created at the center of a 6″ Outer Diameter (OD) copper wound coil. This magnetic field is used to saturate a sample of nanoparticles within the center of the coil for MRX readings. The coil and/or sensing volume may vary in size. The magnetic field strength may be tuned in order to achieve full saturation, and may vary depending on size and/or location of the sample.

The 60 Gauss magnetic field is found to increase the magnetic flux density static offset within the mu-metal shield to a level above the dynamic operating range of the detector sensors (±5 nanoTesla (nT)). The sensors comprise an array of optically pumped magnetometers (OPMs) which were used for magnetocardiography (MCG) measurements. The sensors may not be able to record data after experiencing this large of a static field increase without a lengthy (about 10 seconds) recalibration step, thereby rendering magnetorelaxometry (MRX) measurements impossible.

The above issue is addressed as follows. It is observed that the delta of the magnetic flux density offset settles to near zero asymptotically with repeated pulses of the MRX excitation coil. Once the asymptote is reached, the field offset in the shield is raised by no more than 100 to 200 picoTesla (pT) between pulses. At this point, pulses can be conducted in succession without needing to restart or recalibrate the OPMs, allowing for immediate data recording of magnetic field data after the excitation pulse is shut off.

Because of this phenomenon, prior to any field measurements used for MRX analysis, the shield of the biomagnetic system is pre-conditioned, so that the changes in the magnetization of the shield do not appear as a significant error source when compared to the signal of interest.

The present disclosure provides systems and methods for using a compensation coil in a shielded room or chamber for improving response of optically pumped magnetometers (OPMs) during biomagnetic measurements. This may be used to aid in the recording of MRX measurements. In some embodiments, the compensation coil is used to lower an overall magnetic flux intensity within the sensing area, thereby allowable faster recovery time and/or transient response. In some embodiments, the compensation coil is used to assist in the collection of MRX type readings in a semi-shielded or close-shielded environment. Systems and methods for using a compensation coil may address issues of a long time for the shield and/or sensors to recover after pulses produced during measurements.

Using systems and methods of the present disclosure, a biomagnetic detection platform is constructed that improves the transient response of SERF OPMs. For example, the transient response of the OPMs can be improved by having a lower overall magnetic DC background field (offset) while the sensors are initializing.

A critical factor in detecting MRX samples may be the acquisition of a baseline signal that can be referenced in signal processing. For example, a clinical acquisition may comprise a first acquisition (e.g., baseline and then image) followed by a second acquisition (e.g., baseline, then image, then baseline). In some embodiments, the baseline MRX magnetic field measurement comprises a reference MRX magnetic field measurement performed in absence of paramagnetic or superparamagnetic nanoparticles. In some embodiments, the baseline MRX magnetic field measurement is performed using a baseline spatial configuration that differs from a spatial configuration used to perform the MRX measurement of the magnetic field associated with the individual. In some embodiments, the baseline spatial configuration comprises adjustable spatial positions of the array of one or more optically pumped magnetometers, the individual, and/or a excitation coil.

### Example 3

The systems, methods, devices, and software described herein are used in a number of different applications including in research and healthcare settings, wherein the systems, methods, devices, and software are used to evaluate a status of an individual and in some cases provide a diagnosis for a condition that the individual has. A condition may comprise both an abnormality (including a pre-disease condition) as well as a disease state. Exemplary types of disease evaluated by the systems, methods, devices, and software described herein include cardiac disease, neurologic disease, and gastrointestinal disease.

In some embodiments, devices, systems, software, and methods described herein provide a suggestion for a next diagnostic step to carry out with the individual following sensing and analyzing the EMF of the individual, such as, for example, an additional diagnostic test or modality that will assist in obtaining a diagnosis. Non-limiting examples of diagnostic modalities suggested include imaging, blood testing, and conduction monitoring (e.g., ECG and EEG).

In some embodiments, devices, systems, software, and methods described herein provide a suggestion for a treatment to be provided to an individual following sensing and analyzing the EMF of the individual.

(a) Cardiac Disease

In some embodiments, the systems, methods, devices, and software described herein are used to evaluate an individual for cardiac disease. Non-limiting examples of cardiac disease evaluated by the systems, methods, devices, and software described herein include CAD, arrhythmia, and congestive heart failure.

In some embodiments, the systems, methods, devices, and software described herein are used to evaluate an individual for CAD. In these embodiments, an EMF associated with a heart of an individual is sensed and based on the sensed EMF of the individual, a status of the individual is determined with respect to CAD. In some of these embodiments, a determination is made as to whether coronary disease is present in the individual. In some of these embodiments, a determination is made as to a degree of severity of a CAD that is present. A degree of severity determined, in some embodiments, comprises “severe,” “moderate,” or “mild,” A degree of severity, in some embodiments, comprises a degree of an obstruction of one or more coronary vessels. For example, in some embodiments, an individual may be determined to have >90% obstruction of their Left Anterior Descending (LAD) artery, >80% obstruction of their LAD, >70% obstruction of their LAD, >60% obstruction of their LAD, or >50% obstruction of their LAD. In some embodiments, the systems, methods, devices, and software described herein determine a presence of a pre-CAD state or that a risk of developing coronary artery exists in the individual. For example, in some embodiments, it is determined that an individual has a >90% risk of developing moderate to severe CAD, a >80% risk of developing moderate to severe CAD, a >70% risk of developing moderate to severe CAD, a >60% risk of developing moderate to severe CAD.

In some embodiments, the systems, methods, devices, and software described herein are used in an acute care setting to evaluate individuals with chest pain. For example, in some embodiments, individuals with left sided chest pain of unknown origin are ruled out of having CAD. For example, in some embodiments, individuals with left sided chest pain of unknown origin are ruled in for having CAD. In some embodiments, an individual with a normal ECG and/or at last one normal troponin level is assessed by the systems, devices, methods, and software described herein and determined to either have CAD, not have CAD, have a high likelihood of having CAD, or have a high likelihood of not having CAD.

More specifically, a system as described herein includes at least one EMF sensor (or a plurality of EMF sensors, or a plurality of EMF sensors arranged in an array) that are positioned in proximity to the heart of an individual. In some embodiments the system further comprises shielding to shield the at least one EMF sensor from ambient EMF readings. Once the at least one sensor senses an EMF, the sensed EMF is analyzed by the software described herein including a machine learning algorithm and a determination is made with respect to the status of the heart of the individual. In some embodiments, the analysis process comprises the generation, by the software described herein, of a visual representation of the EMF that is then analyzed. In some embodiments, a sensed EMF that shows a regular pattern without magnetic dipole dispersion, represents a normal finding, an absence of a presence of CAD in the individual, or a low likelihood of a presence of CAD in the individual. In some embodiments, a sensed EMF that shows an irregular pattern of magnetic pole dispersion represents an abnormal finding, a presence of CAD in the individual, or a high likelihood of a presence of CAD in the individual. In some embodiments, a shift in dipole angulation or significant disorganization in the magnetic field map (e.g., a triple pole) indicates a greater degree of vessel stenosis (i.e., greater degree of CAD).

In some embodiments, a suggestion for a treatment is provided. Non-limiting examples of treatments suggested for CAD include conservative treatment (e.g., improve diet and/or exercise), cholesterol lowering treatment, vasodilating medications, rhythm modulating medications, intravascular interventions including stenting, and bypass surgery.

(b) Neurological Disease

In alternative embodiments, the systems, methods, devices, and software described herein are used to evaluate an individual for neurological disease including abnormalities resulting from traumatic injury and stroke. Non-limiting examples of neurological disorders evaluated by the systems, methods, devices, and software described herein include epilepsy, stroke, traumatic brain injury, traumatic spine injury, encephalitis, meningitis, tumor, Alzheimer's disease, Parkinson's disease, ataxia, and psychiatric disorders including schizophrenia, depression, and bipolar disease.

(c) Gastrointestinal Disease

In alternative embodiments, the systems, methods, devices, and software described herein are used to evaluate an individual for gastrointestinal disease including any disease or disorder of any component of the gastrointestinal system including the gastrointestinal tract, the liver (including biliary system), and the pancreas. Non-limiting examples of gastrointestinal disorders evaluated by the systems, methods, devices, and software described herein include gastrointestinal cancers (including tumors of the gastrointestinal tract, liver, and pancreas), Crohn's disease, ulcerative colitis, irritable bowel disease, dismotility disorders, gall stones, colitis, cholangitis, liver failure, pancreatitis, and infections of the gastrointestinal system.

It should be understood, that any device, system, and/or software described herein is configured for use in or is captured by one or more steps of a method.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Particular Implementations

Disclosed is a shielded chamber system for diagnostic evaluation of a condition of an individual. The shielded chamber may comprise an enclosure comprising a plurality of walls, a wall comprising a plurality of layers of magnetic shielding material. The shielded chamber may also comprise one or more application-specific modular components configured to be inserted within the enclosure, wherein an application-specific modular component may comprise an array of biomagnetic field sensors configured to sense an electromagnetic field associated with the individual and generate electromagnetic field data therefrom. The shielded chamber may also comprise one or more holes or passthroughs inserted into at least one wall of the plurality of walls. A hole or passthrough may be configured for passing electrical or data cabling into and out of the enclosure. The wall may comprise two or more layers. each of the two or more layers has a thickness of between 0.1 and 10 millimeters. The wall may comprise a permalloy or a mumetal. The wall comprising a permalloy or a mumetal may be built around a nonmagnetic frame. One of the one or more application-specific modular components may be directed to cardiac applications. One of the one or more application-specific modular components may be a magnetocardiography (“MCG”) module. One of the one or more application-specific modular components may be directed to neurological applications. One of the one or more application-specific modular components may be a magnetoencephalography (“MEG”) module. One of the one or more application-specific modular components may be a module for magnetorelaxometry, employing magnetization coils for site-specific magnetorelaxometry measurements. One of the one or more application-specific modular components may be a module for ultra-low field magnetic resonance imaging (“MRP”) employing magnetization coils to produce an image of the individual. The shielded chamber system may comprise a mounting system comprising the one or more application-specific modular components. The array of biomagnetic field sensors are actuated to create a multi-frame stitched data image. The array of biomagnetic field sensors may comprise at least three biomagnetic field sensors. The array of biomagnetic field sensors may be arranged to match a generalized contour of a portion of a body of the individual. The array of biomagnetic field sensors may comprise optically pumped magnetometer sensors, magnetic induction sensors, magneto-resistive sensors, SQUID sensors, nitrogen vacancy diamonds, fluxgate magnetometers, or a combination thereof. The fluxgate magnetometers comprise Yttrium Iron Garnet film. The shielded chamber system may further comprise a mounting system to insert the one or more application-specific modular components within the enclosure, wherein the mounting system may comprise a magnetic rail system. The shielded chamber system may further comprise a viewing area for patient monitoring. One of the one or more application-specific modular components may be a module for fetal magnetocardiography. One of the one or more application-specific modular components may be a module for fetal magnetoencephalography.

What is claimed is:

1. A shielded chamber system for diagnostic evaluation of a condition of an individual, comprising:
a. an enclosure comprising a plurality of walls, a wall comprising a plurality of layers of magnetic shielding material;
b. one or more application-specific modular components configured to be inserted within the enclosure, wherein an application-specific modular component comprises an array of biomagnetic field sensors configured to sense an electromagnetic field associated with the individual and generate electromagnetic field data therefrom; and
c. one or more holes or passthroughs inserted into at least one wall of the plurality of walls, wherein a hole or passthrough is configured for passing electrical or data cabling into and out of the enclosure.

2. The shielded chamber system of claim 1, wherein the wall comprises two or more layers.

3. The shielded chamber system of claim 2, wherein each of the two or more layers has a thickness of between 0.1 and 10 millimeters.

4. The shielded chamber system of claim 1, wherein the wall comprises a permalloy or a mumetal.

5. The shielded chamber system of claim 4, wherein the wall comprising a permalloy or a mumetal is built around a nonmagnetic frame.

6. The shielded chamber system of claim 1, wherein one of the one or more application-specific modular components is directed to cardiac applications.

7. The shielded chamber system of claim 1, wherein one of the one or more application-specific modular components is a magnetocardiography (“MCG”) module.

8. The shielded chamber system of claim 6, wherein one of the one or more application-specific modular components is directed to neurological applications.

9. The shielded chamber system of claim 8, wherein one of the one or more application-specific modular components is a magnetoencephalography (“MEG”) module.

10. The shielded chamber system of claim 6, wherein one of the one or more application-specific modular components is a module for magnetorelaxometry, employing magnetization coils for site-specific magnetorelaxometry measurements.

11. The shielded chamber system of claim 6, wherein one of the one or more application-specific modular components is a module for ultra-low field magnetic resonance imaging (“MM”) employing magnetization coils to produce an image of the individual.

12. The shielded chamber system of claim 1, wherein the shielded chamber system comprises a mounting system comprising the one or more application-specific modular components.

13. The shielded chamber system of claim 1, wherein the array of biomagnetic field sensors are actuated to create a multi-frame stitched data image.

14. The shielded chamber system of claim 1, wherein the array of biomagnetic field sensors comprises at least three biomagnetic field sensors.

15. The shielded chamber system of claim 1, wherein the array of biomagnetic field sensors is arranged to match a generalized contour of a portion of a body of the individual.

16. The shielded chamber system of claim 1, wherein the array of biomagnetic field sensors comprises optically pumped magnetometer sensors, magnetic induction sensors, magneto-resistive sensors, SQUID sensors, nitrogen vacancy diamonds, fluxgate magnetometers, or a combination thereof.

17. The shielded chamber system of claim 16, wherein the fluxgate magnetometers comprise Yttrium Iron Garnet film.

18. The shielded chamber system of claim 1, further comprising a mounting system to insert the one or more application-specific modular components within the enclosure, wherein the mounting system comprises a magnetic rail system.

19. The shielded chamber system of claim 1, further comprising a viewing area for patient monitoring.

20. The shielded chamber system of claim 6, wherein one of the one or more application-specific modular components is a module for fetal magnetocardiography.

21. The shielded chamber system of claim 8, wherein one of the one or more application-specific modular components is a module for fetal magnetoencephalography.
